(19) United States
(12) Patent Application Publication
(10) Pub. No.: US 2022/0356176 A1
(43) Pub. Date: Nov. 10, 2022

(54) Kv11.1-3.1 INHIBITING METHODS AND COMPOSITIONS

(71) Applicants: The Johns Hopkins University, Baltimore, MD (US); Lieber Institute, Inc., Baltimore, MD (US)

(72) Inventors: Nicholas E. Calcaterra, Baltimore, MD (US); Yifang Huang, Baltimore, MD (US); Pablo DeLeon, Baltimore, MD (US); James Barrow, Baltimore, MD (US)

(21) Appl. No.: 17/617,743

(22) PCT Filed: Jun. 11, 2020
(86) PCT No.: PCT/US2020/037176

§ 371 Date: Dec. 9, 2021

Related U.S. Application Data
(60) Provisional application No. 62/860,305, filed on Jun. 12, 2019.

(51) Int. Cl. C07D 413/12; C07D 401/12; C07D 471/08; C07D 403/12; C07D 487/08; C07D 401/04; C07D 401/14

(52) U.S. Cl. CPC C07D 413/12; C07D 401/12; C07D 471/08; C07D 403/12; C07D 487/08; C07D 401/04; C07D 401/14

(57) ABSTRACT

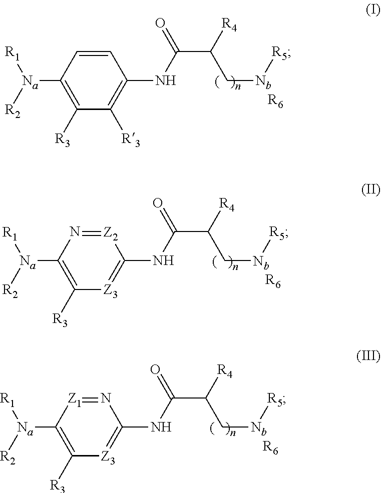

Compounds of formula (I), formula (II), or formula (III) and their use with a neurological or psychiatric disorder, mediated by Kv11.1-3.1 containing potassium channels, including schizophrenia, are disclosed.

### FEDERALLY SPONSORED RESEARCH OR DEVELOPMENT

This invention was made with government support under grant number MH096539 awarded by the National Institutes of Health. The government has certain rights in the invention.

### FIELD OF THE INVENTION

This invention relates to compounds, pharmaceutical compositions and their use for treating neuropsychiatric disorders. In particular, the invention relates to inhibitors of potassium channels containing Kv11.1-3.1 and their use as therapeutics for central nervous system disease.

### BACKGROUND

Schizophrenia is the result of a complex series of neurodevelopmental or other changes that lead to impaired information processing in the brain (Marenco and Weinberger, 2000). No single genetic change, aberrant protein function, or visible brain lesion has been demonstrated to lead to schizophrenia and many different genetic and environmental changes are linked to increased disease risk (Fatemi and Folsom, 2009). While many neurochemical signaling systems, such as the various monoamines, NMDA, and GABA, are likely to play a role in the etiology of schizophrenia (Pickard, 2011), aberrant neuronal firing is the result of these changes and leads to impaired information processing.

With regard to the cognitive impairment, for which no treatment currently exists, patients afflicted with schizophrenia show significant deficits in specific cognitive domains, especially executive function, working memory, and episodic memory. Cognitive domains, which are dysfunctioning in these disorders, are complex functions involving many neurotransmitters and brain regions.

Polymorphisms in the KCNH2 gene have been associated with altered cognitive function and with schizophrenia in multiple independent clinical data sets. See Huffaker et al., 2009; Hashimoto et al., 2013; and Atalar et al., 2010. The KCNH2 gene (also referred to as the human ether-â-go-go related gene or hERG) encodes the protein Kv11.1, which forms voltage-gated potassium channels. See Trudeau et al., 1995; Trudeau et al., 1996. Like other voltage-dependent potassium channels, Kv11.1 has a subunit topology of six transmembrane domains. Four Kv11.1 subunits form a tetramer with a central pore through which ions pass. The risk-associated alleles predict impaired cognitive function in both patients and healthy controls, as well as overexpression in brain of KCNH2-3.1, the truncated transcript, encoding for a Kv11.1 isoform with unique electrophysiological properties (Kv11.1-3.1). See Huffaker et al., 2009.

Kv11.1-3.1 is a primate-specific isoform, enriched in the brain, which lacks the PAS domain critical for the slow-deactivation properties of hERG channels. Rat cortical neurons expressing Kv11.1-3.1-containing Kv11 channels have higher firing rates and faster Kv11 channel deactivation kinetics. See Huffaker et al., 2009. In general, Kv11 channels have been shown to regulate the activity of neurons in multiple brain regions. See Pessia et al., 2008; Ji et al., 2012. Thus, these findings suggest that a possible cause of the cognitive dysfunction in patients with schizophrenia associated with elevated KCNH2-3.1 may be decreased synchrony among functionally connected neurons. See Fano et al., 2012. Indeed, people who carry alleles associated with overexpression of KCNH2-3.1 exhibit more inefficient neuronal processing in the hippocampus and frontal cortex during memory tasks as measured with fMRI. See Huffaker et al., 2009.

Cognitive dysfunction associated with schizophrenia is a major unmet therapeutic need, with important implications for functional outcomes in patients. See Keefe et al., 2012; green et al., 2000. Many risk factors associated with impaired cognition in schizophrenia have been identified, including genes and environmental factors, but their potential role in the development of new therapies has been the subject of few investigations. See Mowry et al., 2013; Svrakic et al., 2013. Recent investigations of KCNH2 in relation to schizophrenia have shown that the genotype associated with increased expression of the KCNH2-3.1 isoform predicts enhanced response to antipsychotic drug therapy, suggesting that targeted modulation of Kv11.1-3.1 channel activity may be a viable drug discovery strategy. See Ji et al., 2012; Apud et al., 2012.

U.S. Pat. No. 8,101,380, for “Schizophrenia-related isoform of KCNH2 and development of antipsychotic drugs,” to Weinberger et al., issued Jan. 24, 2012, which is incorporated by reference in its entirety, is directed to the KCNH2-3.1 isoform, including isolated nucleic acids comprising the polynucleotide sequences identified as encoding the 3.1 isoform, polypeptides having the amino acid sequence encoded by isoform 3.1 nucleic acid molecules, screening assays for finding compounds that can be used to bind to the isoform 3.1 or effect expression or functional activity, and diagnostics predicting the likelihood that an individual will suffer from a neuropsychiatric disorder, e.g., schizophrenia. U.S. Pat. No. 8,871,443, to Weinberger, for “Schizophrenia-related isoform of KCNH2 and development of antipsychotic drugs,” issued Oct. 28, 2014, which is incorporated by reference in its entirety, is directed to methods for predicting the clinical response of a schizophrenic patient to an antipsychotic medication by detecting certain KCNH2 polymorphisms.

### SUMMARY

In some aspects, the presently disclosed subject matter provides a compound of formula (I):

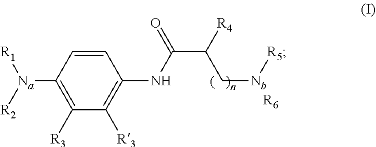

wherein:

n is 0 or 1;

R₁ and R₂ together with nitrogen atom Nₐ to which R₁ and R₂ are bound form an azepanyl or oxazepanyl ring system;

R₃ and R′₃ are each independently H or halogen;

R₄ is H or C₁-C₄ alkyl;

R₅ and R₆ together with nitrogen atom N_{b} to which R₅ and R₆ are bound form a pyrrolidinyl, piperidinyl, oxazepanyl, or azabicyclo[2.2.2]octanyl ring system,

wherein:

the pyrrolidinyl ring system when present is optionally substituted with 4-fluorophenyl in the 3-position;

the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R9)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position;

provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted;

and pharmaceutically acceptable salts thereof.

In other aspects, the presently disclosed subject matter provides a compound of formula (II):

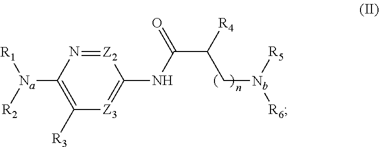

wherein:

n is 0 or 1;

Z₂ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In other aspects, the presently disclosed subject matter provides a compound of formula (III):

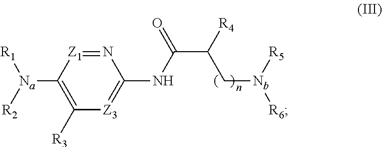

wherein:

n is 0 or 1;

Z₁ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In other aspects, the presently disclosed subject matter provides a pharmaceutical composition comprising at least one compound of formula (I), formula (II), or formula (III), and a pharmaceutically acceptable carrier. In some aspects, the pharmaceutical composition further comprises at least one additional therapeutic agent. In certain aspects, the at least one additional therapeutic agent is selected from the group consisting of one or more antipsychotic agents. In more certain aspects, the one or more antipsychotic agents is selected from olanzapine, risperidone, paliperidone, aripriprazole, clozapine, perphenazine, quetiapine, haloperidol, lurasidone, and combinations thereof.

In yet other aspects, the presently disclosed subject matter provides a method for treating a neurological or psychiatric disorder, or treating symptoms associated with a neurological or psychiatric disorder, the method comprising administering to a subject in need of treatment thereof a therapeutically effective amount of a compound of formula (I), formula (II), or formula (III), or a pharmaceutically acceptable salt thereof:

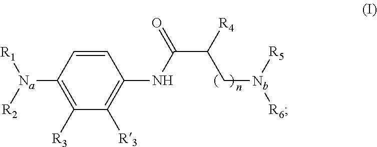

wherein:

n is 0 or 1;

R₁ and R₂ together with nitrogen atom Nₐ to which R₁ and R₂ are bound form an azepanyl or oxazepanyl ring system;

R₃ and R′₃ are each independently H or halogen;

R₄ is H or C₁-C₄ alkyl;

R₅ and R₆ together with nitrogen atom N_{b} to which R₅ and R₆ are bound form a pyrrolidinyl, piperidinyl, oxazepanyl, or azabicyclo[2.2.2]octanyl ring system,

wherein:

the pyrrolidinyl ring system when present is optionally substituted with 4-fluorophenyl in the 3-position;

the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position;

provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted;

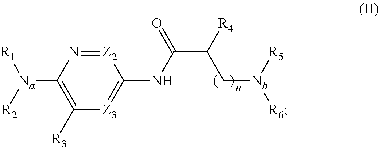

wherein:

n is 0 or 1;

Z₂ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano; or

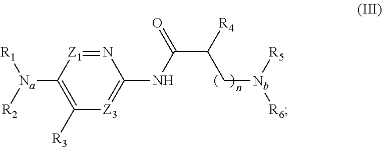

wherein:

n is 0 or 1;

Z₁ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano.

In some aspects, the neurological or psychiatric disorder is selected from schizophrenia, major depression, a depressive phase of bipolar disorder, attention deficit disorder, attention deficit/hyperactivity disorder, substance dependency, and increased appetite associated with smoking cessation or antipsychotic use. In certain aspects, the neurological or psychiatric disorder is schizophrenia.

In particular aspects, administering to the subject a therapeutically effective amount of a compound of formula (I), formula (II), or formula (III), or a pharmaceutically acceptable salt thereof, inhibits one or more Kv11.1-3.1 containing potassium channels.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### DETAILED DESCRIPTION

The presently disclosed subject matter now will be described more fully hereinafter with reference to the accompanying Examples, in which some, but not all embodiments of the inventions are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Examples. Therefore, it is to be understood that the presently disclosed subject matter is not to be limited to the specific embodiments disclosed and that modifications and other embodiments are intended to be included within the scope of the appended claims.

### I. Definitions

The terms below, when used herein, have the following meanings unless indicated otherwise.

As used in this specification and the appended claims, the singular forms “a,” “an,” and “the” include plural references unless the content clearly dictates otherwise.

When any variable (e.g., aryl, heterocycle, R₁, and the like) occurs more than one time in any constituent, its definition on each occurrence is independent at every other occurrence.

“Alkyl” refers to a saturated hydrocarbon chain. Such hydrocarbon chains may be branched or linear. “Alkyl” groups may be substituted or unsubstituted. Exemplary substitutions include, but are not limited to, halogen, amido, aryl or alkoxyl.

The term “C₁-C₄” (for example), or “C₁-₄”, includes, for this example, an alkyl group containing 4, 3, 2, or 1 carbon atom(s). “Alkoxyl” refers to the group —OR, where R includes “C₁-C₆ alkyl”, “C₃-C₈ cycloalkyl”, “heterocycloalkyl”, “aryl”, “heteroaryl”, “aralkyl” or “heteroarylalkyl”. C₁-C₄ alkoxyl refers to the group —OR where R is a C₁-C₄ alkyl group. Aryloxyl refers to the group —OR where R is an aryl group.

“Ring” refers to a cyclic structure formed by the joining of two substituents. Rings can be saturated or unsaturated, aliphatic or aromatic. In one embodiment, a ring contains zero heteroatoms, i.e., a carbocycle. In other embodiments, rings can contain one or two heteroatoms, i.e., heterocycles.

The term “heterocycle” or “heterocyclic” includes heteroaryl moieties. Nonlimiting examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyI, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydro isoquinolinyl, tetrahydroquinolinyl, thiamorpholinyl, thiamorpholinyl sulfoxide, thiazolyl, thiazolinyl, thienofuryl, thienothienyl, thienyl and triazolyl.

In certain embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 [pi] electrons shared in a cyclic array; and having, in addition to carbon atoms, between one and about three heteroatoms selected from the group consisting of N, O, and S which may be saturated, such as piperidinyl, partially saturated, or unsaturated, such as pyridinyl, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

In certain other embodiments, the heterocyclic group is fused to an aryl or heteroaryl group. Examples of such fused heterocycles include, without limitation, tetrahydroquinolinyl and dihydrobenzofuranyl. Examples of heterocycloalkyls include, without limitation, azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

“Halogen” and “halo” refer to fluorine, chlorine, bromine and iodine.

A moiety that is “substituted” is one in which one or more hydrogens have been independently replaced with another chemical substituent. Suitable substituents include, without limitation, halogen, hydroxy, oxo (e.g., an annular —CH— substituted with oxo is —C(═O)—), nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

Other possible substituents, which are themselves not further substituted (unless expressly stated otherwise) are: (a) halo, cyano, oxo, carboxy, formyl, nitro, amino, amidino, and guanidino, and (b) C₁-C₆ alkyl or alkenyl or arylalkyl imino, carbamoyl, azido, carboxamido, mercapto, hydroxy, hydroxyalkyl, alkylaryl, arylalkyl, C₁-C₈ alkyl, SO₂CF₃, CF₃, SO₂Me, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₂-C₁₅ N,N-alkylcarbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, or aryl, wherein each of the foregoing in (b) can be further substituted with one more moieties listed in (a), above. Each instance of C₈ noted in this paragraph may be, in further embodiments, C₆.

The presently disclosed compounds may contain one or more asymmetric centers and may thus occur as racemates, racemic mixtures, single enantiomers, diastereomeric mixtures, and individual diastereomers.

It will be understood that, as used herein, references to the presently disclosed compounds are meant to also include the pharmaceutically acceptable salts, and also salts that are not pharmaceutically acceptable when they are used as precursors to the free compounds or in other synthetic manipulations.

The presently disclosed compounds may be administered in the form of a pharmaceutically acceptable salt. The term “pharmaceutically acceptable salts” refers to salts prepared from pharmaceutically acceptable non-toxic bases or acids. When the presently disclosed compound is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines, such as naturally occurring and synthesized substituted amines. Other pharmaceutically acceptable organic non-toxic bases from which salts can be formed include ion exchange resins, such as, for example, arginine, betaine, caffeine, choline, N, N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamme, isopropylamine, lysine, methylglucamine, morpholine, piperazine, piperidine, polyamine resins, procaine, purines, theobromine, triethylamine, trimethylamine, tripropylamine, and tromethamine.

When the presently disclosed compound is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

A “pharmaceutically acceptable excipient” or “pharmaceutically acceptable carrier” refers to an excipient that can be included in the presently disclosed compositions and that causes no significant adverse toxicological effects to the subject or patient to which the composition is administered. “Pharmacologically effective amount,” “physiologically effective amount,” and “therapeutically effective amount” are used interchangeably herein to mean the amount of an active agent present in a pharmaceutical preparation that is needed to provide a desired level of active agent and/or conjugate in the bloodstream or in the target tissue. The precise amount will depend upon numerous factors, e.g., the particular active agent, the components and physical characteristics of pharmaceutical preparation, intended patient population, patient considerations, and the like, and can readily be determined by one skilled in the art, based upon the information provided herein and available in the relevant literature.

The terms “patient” and “subject” are used interchangeably and refer to a living organism suffering from or prone to a condition that can be prevented or treated by administration of an active agent as described herein, and includes both humans and animals. In one embodiment, the patient is a human patient.

The term “mammal” “mammalian” or “mammals” includes humans, as well as animals, such as dogs, cats, horses, pigs and cattle.

Without being bound by any one particular theory, the administration of presently disclosed compounds in an “effective amount” or “therapeutically effective amount” provides a concentration of the compound that provides a clinical benefit to the patient. Without being bound by theory, the clinical benefit is believed to result from the inhibition of KCNH2-3.1 or ion channels containing the same. “Treating” or “treatment” of a disease state includes: 1) preventing the disease state, i.e., causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) attenuating the disease state, i.e., reducing the number or intensity of one or more symptoms associated with the disease state, such that one or more symptoms is reduced but may, or may not be completely eliminated; and/or 4) relieving the disease state, i.e., causing temporary or permanent regression of the disease state or its clinical symptoms.

### II. Compounds Of Formula (I), Formula (II), and Formula (III)

Disclosed herein are novel benzimidazole derivatives which, preferably, are inhibitors of the Kv11.1-3.1 containing potassium channel, and which are useful in the treatment or prevention of neurological or psychiatric disorders or diseases in which Kv11.1-3.1 is involved or for which inhibition or attenuation of Kv11.1-3.1 contributes to a therapeutic benefit. The presently disclosed compounds are characterized by their activity to inhibit the ion channel produced from the KCNH2-3.1 transcript. In some embodiments, the presently disclosed compounds are effective to inhibit Kv11.1-3.1 containing ion channels in an assay which determines the inhibitory concentration (IC₅₀) for the transmission of thallium through the channel as described herein and have an IC₅₀ superior or equal to 10 μM. In some embodiments, the ICso is superior or equal to 2 μM. In some embodiments, the pIC₅₀ is superior or equal to 500 nM.

The ability of the presently disclosed compounds to inhibit the activity of the ion channel produced from the KCNH2-3.1 transcript may be determined by methods known to those in the art for measuring ion channel inhibition. One method for measuring ion channel activity uses a thallium flux assay (Titus et al., 2009; FluxOR™ kit from Life Technologies). This assay measures the passage of thallium through the channel as indicated by a thallium sensitive dye loaded into the cell. Using this assay preferred compounds have an ICso superior or equal to 10 μM. In increasingly preferred embodiments, the IC₅₀ as so determined is superior or equal to 2 μM. In a more preferred embodiment, the pIC₅₀ as so determined is superior or equal to 500 nM.

In some embodiments, the presently disclosed subject matter provides a compound of formula (I):

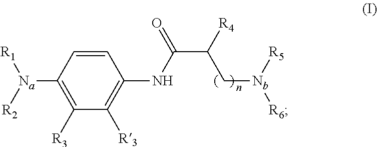

wherein:

n is 0 or 1;

R₁ and R₂ together with nitrogen atom Nₐ to which R₁ and R₂ are bound form an azepanyl or oxazepanyl ring system;

R₃ and R′₃ are each independently H or halogen;

R₄ is H or C₁-C₄ alkyl;

R₅ and R₆ together with nitrogen atom N_{b} to which R₅ and R₆ are bound form a pyrrolidinyl, piperidinyl, oxazepanyl, or azabicyclo[2.2.2]octanyl ring system,

wherein:

the pyrrolidinyl ring system when present is optionally substituted with 4-fluorophenyl in the 3-position;

the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position;

provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted;

and pharmaceutically acceptable salts thereof.

In some embodiments, the presently disclosed subject matter provides a compound of formula (II):

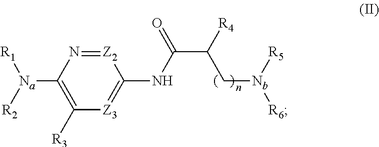

wherein:

n is 0 or 1;

Z₂ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In some embodiments, the presently disclosed subject matter provides a compound of formula (III):

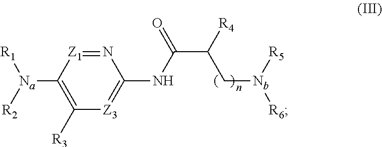

wherein:

n is 0 or 1;

Z₁ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In some embodiments, the compound is a compound of formula (II) or formula (III), and wherein R₂ is cyclohexyl or phenyl.

In some embodiments, the compound is a compound of formula (II) or formula (III), and R₁ and R₂ together with nitrogen atom Nₐ to which R₁ and R₂ are bound form a 6-, 7-, 8-, or 9-membered saturated cyclic, heterocyclic, or bicyclic ring system selected from azepanyl, oxazepanyl, azabicyclo[3.2.1]octanyl, and azabicyclo[3.2.2.]nonanyl.

In some embodiments, the compound is a compound of formula (II) or formula (III), and R₅ and R₆ together with nitrogen atom N_{b} to which R₅ and R₆ are bound form a 5-, 6-, 7-, or 8-membered saturated cyclic, heterocyclic, or bicyclic ring system selected from pyrrolidinyl, piperidinyl, oxazepanyl, azabicyclo[2.2.1]heptanyl, azabicyclo[3.2.1]octanyl, and azabicyclo[2.2.2]octanyl, wherein the pyrrolidinyl and piperidinyl can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano.

In some embodiments, the compound of formula (I) has the following structure:

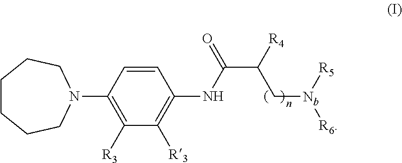

In yet other embodiments, the compound of formula (I) has the following structure:

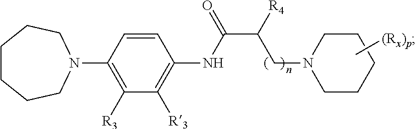

wherein p is 0, 1, or 2 subject to the provisions below;

wherein the piperidinyl ring system is substituted by one or two Rx with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position.

In particular embodiments, the compound is a compound of formula (I) selected from the following:

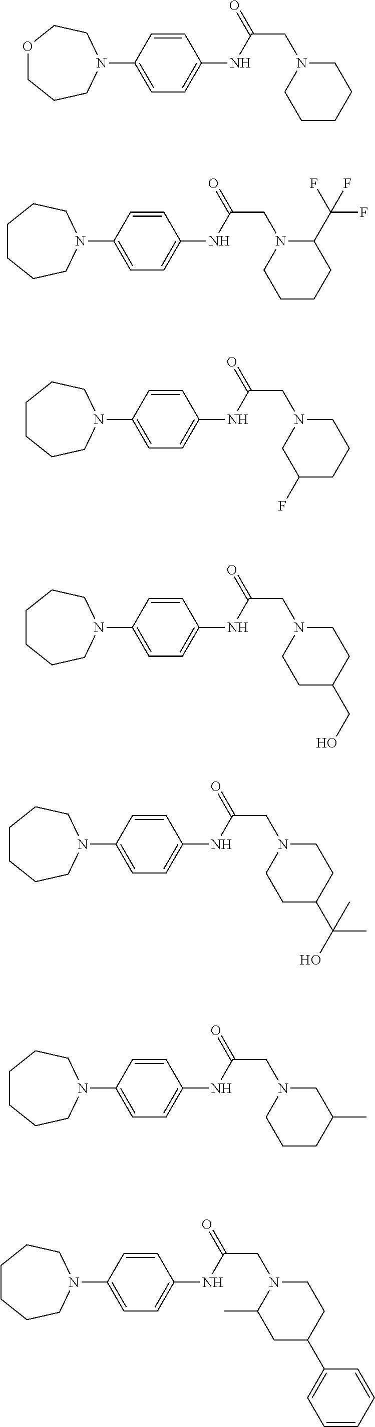

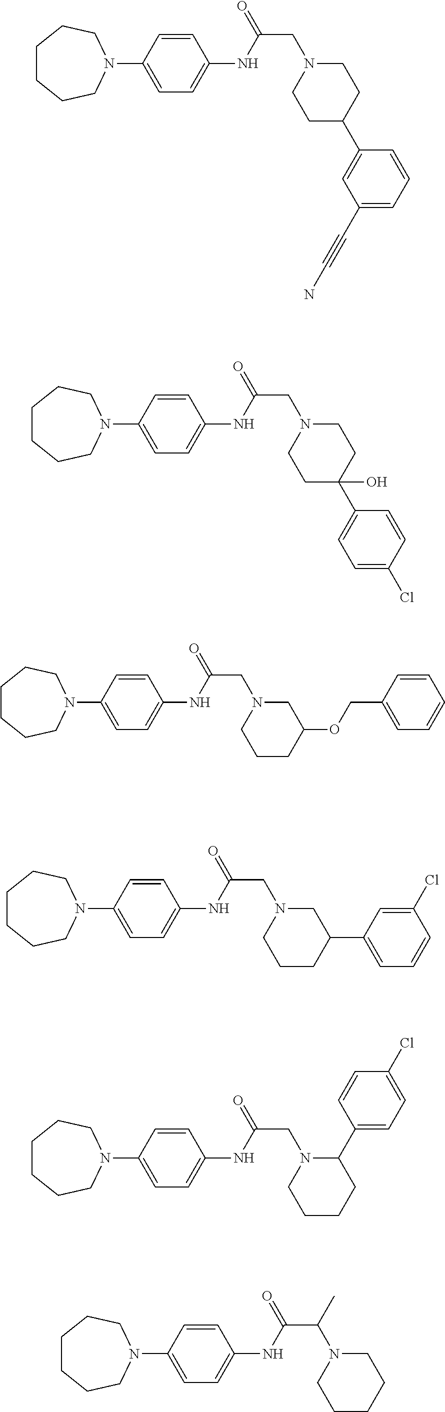 [SMILES: NC#Cc1cccc(C2CCN(CC(=O)Nc3ccc(N4CCCCCC4)cc3)CC2)c1 ; O=C(CN1CCC(O)(c2ccc(Cl)cc2)CC1)Nc1ccc(N2CCCCCC2)cc1 ; O=C(CN1CCCC(OCc2ccccc2)C1)Nc1ccc(N2CCCCCC2)cc1 ; O=C(CN1CCCC(c2cccc(Cl)c2)C1)Nc1ccc(N2CCCCCC2)cc1 ; O=C(CN1CCCCC1c1ccc(Cl)cc1)Nc1ccc(N2CCCCCC2)cc1 ; CC(C(=O)Nc1ccc(N2CCCCCC2)cc1)N1CCCCC1]

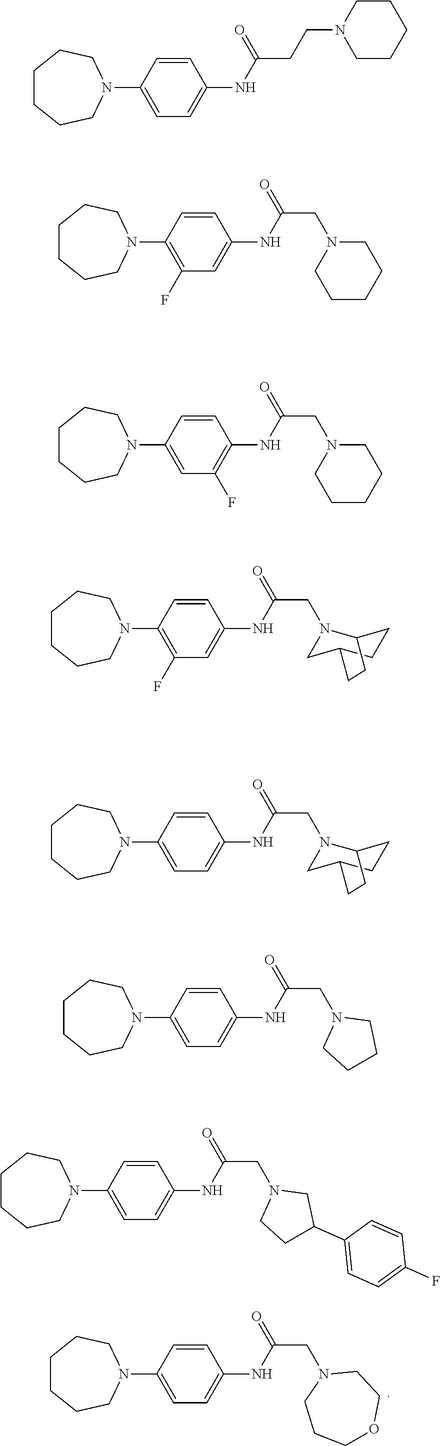

In some embodiments, the compound is a compound of formula (II) and:

n is 0 or 1;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl; or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In some embodiments of the compound of formula (II):

n is 0;

R₂ is cyclohexyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, selected from piperidinyl, azabicyclo[3.2.1]octanyl, and azabicyclo[3.2.2]nonanyl, wherein the piperidinyl ring system when present can optionally be substituted with C₁-C₄ alkyl;

R₄ is H;

R₅ is H or C₁-C₄ alkyl;

R₆ is selected from cyclohexyl, cycloheptyl, phenyl, and benzyl; or R₅ and R₆ together with nitrogen atom N_{b} to which R₅ and R₆ are bound form a 6-, 7-, or 8-membered saturated cyclic, heterocyclic, or bicyclic ring system selected from piperidinyl, azabicyclo[3.2.1]octanyl, azabicyclo[2.2.2]octanyl, and azabicyclo[2.2.1]heptanyl.

In particular embodiments of a compound of formula (II):

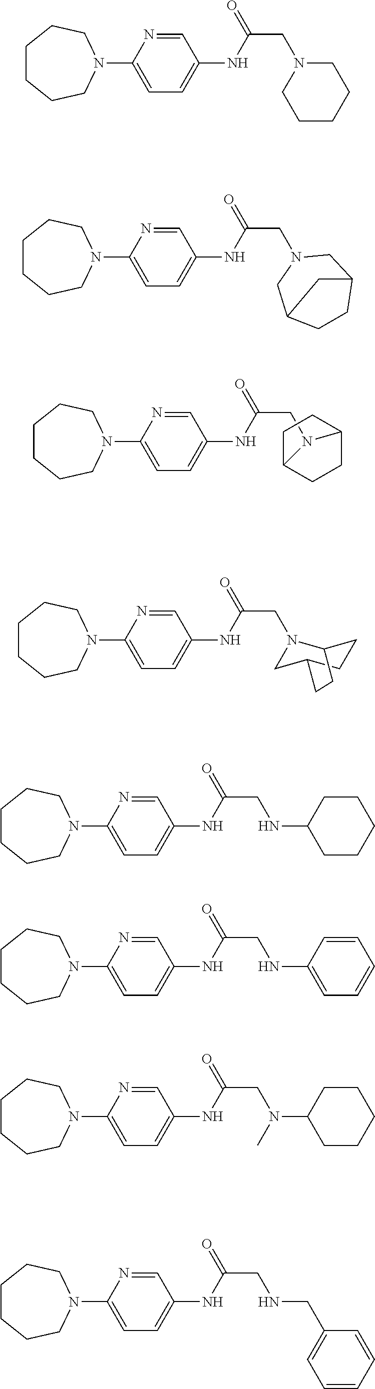

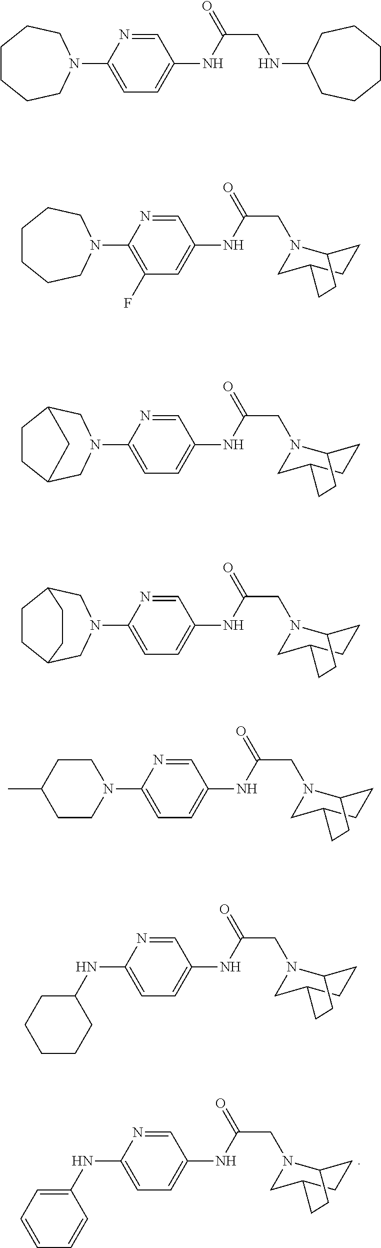

In some embodiments of a compound of formula (III):

n is 0 or 1;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In some embodiments of a compound of formula (III):

n is 1;

R₁ and R₂ together with nitrogen atom Nₐ to which R₁ and R₂ are bound form an azepanyl ring system;

R₃ and R₄ are each H;

R₅ and R₆ together with nitrogen atom N_{b} to which R₅ and R₆ are bound form a piperidinyl ring system; and the compound is selected from:

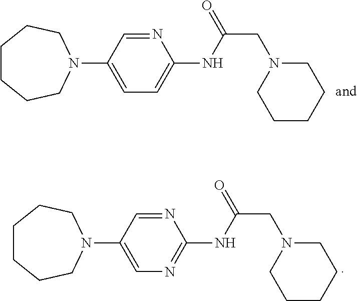 [SMILES: O=C(CN1CCCCC1)Nc1ccc(N2CCCCCC2)cn1 ; O=C(CN1CCCCC1)Nc1ncc(N2CCCCCC2)cn1]

In other embodiments, the presently disclosed subject matter provides a pharmaceutical composition comprising at least one compound of formula (I), formula (II), or formula (III), and a pharmaceutically acceptable carrier. In certain embodiments, the pharmaceutical composition further comprises at least one additional therapeutic agent. In more certain embodiments, the at least one additional therapeutic agent is selected from the group consisting of one or more antipsychotic agents. In yet more certain embodiments, the one or more antipsychotic agents is selected from olanzapine, risperidone, paliperidone, aripriprazole, clozapine, perphenazine, quetiapine, haloperidol, lurasidone, and combinations thereof.

### III. Methods of Treating a Neurological or Psychiatric Disorder or Symptoms Associated with Thereof

In some embodiments, the presently disclosed subject matter provides a method for treating or preventing a neurological or psychiatric disorder, or treating symptoms associated with a neurological or psychiatric disorder, and in particular such disorders for which inhibiting potassium channels containing the Kv11.1-3.1 isoform (referred to herein as “Kv11.1-3.1 containing potassium channels”) provides a therapeutic effect. A functional Kv11 potassium channel may contain various Kv11 isoforms, so long as the total amount of subunits is four. For example, the channel may contain Kv11.1-1A:Kv11.1-3.1 heteromers. Without being bound by any particular theory, it is thought that the presently disclosed compounds of formula (I), formula (II), or formula (III) inhibit the flow of potassium ions through the ion channel (formed by the tetramer) by one or more mechanisms including, for example, blockage of the channel pore or interfering with the proteins that form the tetramer, thereby interfering with the channel function. Accordingly, in one embodiment, the presently disclosed subject matter provides methods for treating and/or preventing a disease, condition, or disorder for which inhibiting Kv11.1-3.1-containing potassium channels provides a beneficial therapeutic effect.

In another embodiment, the presently disclosed subject matter provides a method for inhibiting Kv11.1-3.1 containing potassium channels in a subject by administering one or more compounds of formula (I), formula (II), or formula (III). These method comprises administering to a subject in need thereof an effective amount of a compound of formula (I), formula (II), or formula (III), or a pharmaceutically acceptable salt thereof:

Accordingly, in some embodiments, the presently disclosed subject matter provides a method for treating a neurological or psychiatric disorder, or treating symptoms associated with a neurological or psychiatric disorder, the method comprising administering to a subject in need of treatment thereof a therapeutically effective amount of a compound of formula (I), formula (II), or formula (III), or a pharmaceutically acceptable salt thereof:

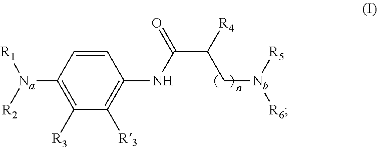

wherein:

n is 0 or 1;

R₁ and R₂ together with nitrogen atom Nₐ to which R₁ and R₂ are bound form an azepanyl or oxazepanyl ring system;

R₃ and R′₃ are each independently H or halogen;

R₄ is H or C₁-C₄ alkyl;

R₅ and R₆ together with nitrogen atom N_{b} to which R₅ and R₆ are bound form a pyrrolidinyl, piperidinyl, oxazepanyl, or azabicyclo[2.2.2]octanyl ring system,

wherein:

the pyrrolidinyl ring system when present is optionally substituted with 4-fluorophenyl in the 3-position;

the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position;

provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted;

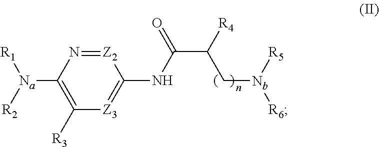

wherein:

n is 0 or 1;

Z₂ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano; or

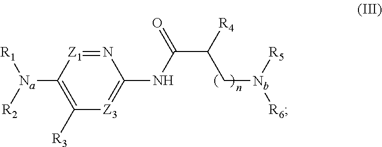

wherein:

n is 0 or 1;

Z₁ and Z₃ are each independently N or CR₇, wherein R₇ is H or halogen;

R₁ is H;

R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;

R₃ is H or halogen;

R₄ and R₅ are each independently H or C₁-C₄ alkyl;

R₆ is selected from cycloalkyl, phenyl, and benzyl;

or R₅ and R₆ together with nitrogen atom N_{b} to which R₅ and R₆ are bound form a 5-, 6-, 7-, or 8-membered saturated cyclic, heterocyclic, or bicyclic ring system,

wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;

and pharmaceutically acceptable salts thereof.

In certain embodiments, the neurological or psychiatric disorder is selected from schizophrenia, major depression, a depressive phase of bipolar disorder, attention deficit disorder, attention deficit/hyperactivity disorder, substance dependency, and increased appetite associated with smoking cessation or antipsychotic use. In more certain embodiments, the neurological or psychiatric disorder is schizophrenia.

In some embodiments, administering to the subject a therapeutically effective amount of a compound of formula (I), formula (II), or formula (III), or a pharmaceutically acceptable salt thereof, inhibits one or more Kv11.1-3.1 containing potassium channels.

The compounds for use in the instant method may be selected from any one or any combination of compounds designated 1-39 herein.

For use in the method, the presently disclosed compound or compounds described above, is/are typically provided as a pharmaceutical composition wherein the compound or compounds is/are present in combination with a pharmaceutically acceptable carrier as described herein. Such pharmaceutical compositions also are provided by this disclosure.

For use in the presently disclosed methods, the presently disclosed compound(s) described hereinabove, also may be used in combination with another additional therapeutic agent.

The presently disclosed methods may be used to treat or prevent a neurological or psychiatric disorder. In particular, exemplary embodiments include methods of treating or preventing schizophrenia, major depression, a depressive phase of bipolar disorder, attention deficit disorder, attention deficit/hyperactivity disorder, substance dependency, or increased appetite associated with smoking cessation or antipsychotic use.

### IV. Indications

As provided hereinabove, the presently disclosed compounds of formula (I), formula (II), or formula (III) can be used for treating neurological and psychiatric disease, and, in a particular embodiment, neuropsychiatric diseases for which inhibiting Kv11.1-3.1 containing potassium channels provides a therapeutic benefit.

Significant psychiatric indications, as discussed above, include, but are not limited to, ADHD, obsessive-compulsive disorder, alcoholism and other addictions, depression, bipolar disorder, age-associated cognitive symptoms, impulse control disorders, including compulsive gambling, sexual behavior, and other compulsive destructive behaviors, in particular, schizophrenia.

In one embodiment, a method for treating conditions in which inhibition of Kv11.1-3.1 containing potassium channels is beneficial comprises administering to a patient in need thereof a compound of formula (I), formula (II), or formula (III) described hereinabove for use in the present methods.

In one specific embodiment, a method for treating schizophrenia or psychosis comprises administering a compound of formula (I), formula (II), or formula (III) that inhibits Kv11.1-3.1 containing potassium channels described hereinabove. In another specific embodiment, a method for treating schizophrenia or psychosis comprises administering to a patient in need thereof a pharmaceutical composition comprising a compound of formula (I), formula (II), or formula (III) that inhibits Kv11.1-3.1 containing potassium channels described hereinabove.

The Diagnostic and Statistical Manual of Mental Disorders (DSM-IV-TR) (2000, American Psychiatric Association, Washington, D.C.) provides a diagnostic tool that includes paranoid, disorganized, catatonic or undifferentiated schizophrenia and substance-induced psychotic disorders. As used herein, the term “schizophrenia or psychosis” includes the diagnosis and classification of these mental disorders as described in DSM-IV-TR and the term is intended to include similar disorders described in other sources. Disorders and conditions encompassed herein include, but are not limited to, conditions or diseases, such as schizophrenia or psychosis. Among the forms of schizophrenia which may be treated with the presently disclosed compounds and methods are paranoid, disorganized, catatonic, undifferentiated, or residual type, schizophreniform disorder, schizoaffective disorder, for example of the delusional type or the depressive type, delusional disorder, psychotic disorder, brief psychotic disorder, shared psychotic disorder, psychotic disorder due to a general medical condition and substance-induced or drug-induced (for example psychosis induced by alcohol, amphetamine, cannabis, cocaine, hallucinogens, inhalants, opioids, phencyclidine, ketamine and other dissociative anaesthetics, and other psychostimulants), psychosis/psychotic disorder, psychosis associated with affective disorders, brief reactive psychosis, schizoaffective psychosis, “schizophrenia-spectrum” disorders, such as schizoid or schizotypal personality disorders, personality disorder of the paranoid type, personality disorder of the schizoid type, illness associated with psychosis (such as major depression, manic depressive (bipolar) disorder, Alzheimer's disease and post- traumatic stress syndrome), including both the positive and the negative symptoms of schizophrenia and other psychoses.

In another embodiment, the presently disclosed subject matter provides a method for treating cognitive disorders comprising administering to a patient in need thereof one or more compounds of formula (I), formula (II), or formula (III) described hereinabove that inhibit Kv11.1-3.1 containing potassium channels. The DSM-IV-TR also provides a diagnostic tool that includes cognitive disorders including dementia, delirium, amnestic disorders and age-related cognitive decline. As used herein, the term “cognitive disorders” includes the diagnosis and classification of these disorders as described in DSM-IV-TR and the term is intended to include similar disorders described in other sources. Disorders and conditions encompassed herein include, but are not limited to, disorders that comprise as a symptom a deficiency in attention and/or cognition, such as dementia (associated with Alzheimer's disease, ischemia, multi-infarct dementia, trauma, intracranial tumors, cerebral trauma, vascular problems or stroke, alcoholic dementia or other drug-related dementia, AIDS, HIV disease, Parkinson's disease, Huntington's disease, Pick's disease, Creutzfeldt Jacob disease, perinatal hypoxia, other general medical conditions or substance abuse), Alzheimer's disease, mild cognitive impairment, multi-infarct dementia, Lewy body dementia, AIDS-related dementia, and Frontotemporal dementia, delirium, amnestic disorders or age-related cognitive decline.

In another embodiment, the presently disclosed subject matter provides a method for treating anxiety disorders comprising administering to a patient in need thereof one or more compounds of formula (I), formula (II), or formula (III) described hereinabove that inhibit Kv11.1-3.1 containing potassium channels. The DSM-IV-TR also provides a diagnostic tool that includes anxiety disorders as generalized anxiety disorder, obsessive-compulsive disorder and panic attack. As used herein, the term “anxiety disorders” includes the diagnosis and classification of these mental disorders as described in DSM-IV-TR and the term is intended to include similar disorders described in other sources. Disorders and conditions encompassed herein include, but are not limited to, anxiety disorders, such as, acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition.

In another specific embodiment, a method for treating mood and depressive disorders comprises administering to a patient in need thereof one or more compounds described hereinabove that inhibit Kv11.1-3.1 containing potassium channels. As used herein, the term “mood and depressive disorders” includes the diagnosis and classification of these medical conditions and disorders described in the DSM-IV-TR and the term is intended to include similar disorders described in other sources. Disorders and conditions encompassed herein include, but are not limited to, bipolar disorders, mood disorders including depressive disorders, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression; major depressive disorder, dysthymic disorder, minor depressive disorder, premenstrual dysphoric disorder, post-psychotic depressive disorder of schizophrenia, a major depressive disorder superimposed on a psychotic disorder, such as delusional disorder or schizophrenia, a bipolar disorder, for example, bipolar I disorder, bipolar II disorder, cyclothymic disorder, depression including unipolar depression, seasonal depression and post-partum depression, premenstrual syndrome (PMS) and premenstrual dysphoric disorder (PDD), mood disorders due to a general medical condition, and substance-induced mood disorders.

In other embodiments, the compounds of formula (I), formula (II), or formula (III) described hereinabove that inhibit Kv11.1-3.1 containing potassium channels can be used to treat other types of cognitive, learning and mental related disorders including, but not limited to, learning disorders, such as a reading disorder, a mathematics disorder, or a disorder of written expression, attention-deficit/hyperactivity disorder, age-related cognitive decline, pervasive developmental disorder including autistic disorder, attention disorders, such as attention-deficit hyperactivity disorder (ADHD) and conduct disorder; an NMDA receptor-related disorder, such as autism, depression, benign forgetfulness, childhood learning disorders and closed head injury; a neurodegenerative disorder or condition, such as neurodegeneration associated with cerebral trauma, stroke, cerebral infarct, epileptic seizure, neurotoxin poisoning, or hypoglycemia-induced neurodegeneration; multi-system atrophy; movement disorders, such as akinesias and akinetic-rigid syndromes (including, Parkinson's disease, drug-induced parkinsonism, post-encephalitic parkinsonism, progressive supranuclear palsy, multiple system atrophy, corticobasal degeneration, parkinsonism-ALS dementia complex and basal ganglia calcification), medication-induced parkinsonism (such as, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Huntington's disease, dyskinesia associated with dopamine agonist therapy, Gilles de la Tourette's syndrome, epilepsy, muscular spasms and disorders associated with muscular spasticity or weakness including tremors; dyskinesias, including tremor (such as, rest tremor, postural tremor, intention tremor and essential tremor), restless leg syndrome, chorea (such as Sydenham's chorea, Huntington's disease, benign hereditary chorea, neuroacanthocytosis, symptomatic chorea, drug-induced chorea and hemiballism), myoclonus (including, generalized myoclonus and focal myoclonus), tics (including, simple tics, complex tics and symptomatic tics), dystonia (including, generalized, idiopathic, drug-induced, symptomatic, paroxysmal, and focal (such as blepharospasm, oromandibular, spasmodic, spasmodic torticollis, axial dystonia, hemiplegic and dystonic writer's cramp); urinary incontinence; neuronal damage (including ocular damage, retinopathy or macular degeneration of the eye, tinnitus, hearing impairment and loss, and brain edema); emesis; and sleep disorders, including insomnia and narcolepsy.

Of the disorders above, the treatment of schizophrenia, bipolar disorder, depression, including unipolar depression, seasonal depression and post-partum depression, cognitive disorders associated with dementia, AIDS dementia, Alzheimer's, Parkinson's, Huntington's disease, spasticity, myoclonus, muscle spasm, tinnitus and hearing impairment and loss are of particular importance.

In yet other particularly desirable embodiments, the compounds of formula (I), formula (II), or formula (III) described hereinabove that inhibit Kv11.1-3.1 containing potassium channels are useful for treating mild cognitive impairment. Accordingly, a method for treating mild cognitive impairment comprises administering to a patient in need thereof one or more compounds of formula (I), formula (II), or formula (III) described hereinabove that inhibit Kv11.1-3.1 containing potassium channels.

In still other particularly desirable embodiments, the compounds of formula (I), formula (II), or formula (III) described hereinabove that inhibit Kv11.1-3.1 containing potassium channels are useful for improving cognitive deficits associated with cerebral trauma (i.e., traumatic brain injury). Accordingly, a method for treating cognitive deficits associated with cerebral trauma (i.e., traumatic brain injury) comprises administering to a patient in need thereof one or more compounds of formula (I), formula (II), or formula (III) described hereinabove that inhibit Kv11.1-3.1 containing potassium channels.

In further particularly desirable embodiments, the compounds of formula (I), formula (II), or formula (III) described hereinabove that inhibit Kv11.1-3.1 containing potassium channels are useful for treating schizophrenia. Accordingly, a method for treating schizophrenia comprises administering to a patient in need thereof one or more compounds of formula (I), formula (II), or formula (III) described hereinabove that inhibit Kv11.1-3.1 containing potassium channels.

The compounds of formula (I), formula (II), or formula (III) described hereinabove that inhibit Kv11.1-3.1 containing potassium channels, are useful in methods for the prevention, treatment, control, amelioration, or reduction of risk of the diseases, disorders and conditions noted herein.

### V. Combination Therapies

Compounds of formula (I), formula (II), or formula (III) described hereinabove that inhibit Kv11.1-3.1 containing potassium channels are further useful in a method for the prevention or treatment of the aforementioned diseases, disorders and conditions in combination with other therapeutic agents. In many instances, the combination of the drugs together is safer or more effective than either drug alone; the presently disclosed compounds and the other active ingredients may often be used in lower doses than when each is used singly. The drug(s) in the combination may be administered contemporaneously or sequentially (i.e., one preceding or following the other, at any appropriate time interval). When administered contemporaneously, the drugs may be administered separately, or a single dosage form may contain both active agents.

Accordingly, the presently disclosed compounds may be used in combination with other agents which are known to be beneficial in the subject indications, or other drugs that affect receptors or enzymes that either increase the efficacy, safety, convenience, or reduce unwanted side effects or toxicity of the presently disclosed compounds. It will be appreciated that any of the drugs listed herein may be in the form of a pharmaceutically acceptable salt.

The term “combination” is used in its broadest sense and means that a subject is administered at least two agents, more particularly a compound of formula (I), formula (II), or formula (III), i.e., an “active agent,” and one or more agents as provided herein. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I), formula (II), or formula (III) described herein can be administered alone or in combination with adjuvants that enhance stability of the compounds of formula (I), formula (II), or formula (III), alone or in combination with one or more antibacterial agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I), formula (II), or formula (III) and at least one additional therapeutic agent can be varied so long as the beneficial effects of the combination of these agents are achieved. Accordingly, the phrase “in combination with” refers to the administration of a compound of formula (I), formula (II), or formula (III) and at least one additional therapeutic agent either simultaneously, sequentially, or a combination thereof. Therefore, a subject administered a combination of a compound of formula (I), formula (II), or formula (III) and at least one additional therapeutic agent can receive compound of formula (I), formula (II), or formula (III) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I), formula (II), or formula (III) and at least one additional therapeutic agent are administered simultaneously, they can be administered to the subject as separate pharmaceutical compositions, each comprising either a compound of formula (I), formula (II), or formula (III) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I), formula (II), or formula (III) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher than what can be expected based on the observed activities of the individual components when used alone. Further, a “synergistically effective amount” of a component refers to the amount of the component necessary to elicit a synergistic effect in, for example, another therapeutic agent present in the composition.

In a particularly preferred embodiment, the subject compound is employed in combination with levodopa, with or without a selective extracerebral decarboxylase inhibitor, such as carbidopa or benserazide. In other embodiments, the presently disclosed Kv11.1-3.1 containing potassium channel inhibitor is administered in combination with anticholinergics, such as biperiden and trihexyphenidyl (benzhexol) hydrochloride, COMT inhibitors, such as tolcapone, entacapone and opicapone, MOA-B inhibitors, antioxidants, A2a adenosine receptor antagonists, cholinergic agonists, NMDA receptor antagonists, serotonin receptor antagonists and dopamine receptor agonists, such as alentemol, bromocriptine, fenoldopam, lisuride, naxagolide, pergolide and pramipexole.

In another embodiment, the subject compound may be employed in combination with a neuroleptic or antipsychotic agent, or pharmaceutically acceptable salts thereof. Classes of neuroleptic agents include phenothiazines; thioxanthenes; heterocyclic dibenzazepines; butyrophenones; diphenylbutylpiperidines; indolones, such as acepromazine, amisulpride, amoxapine, aripiprazole, asenapine, benperidol, bifeprunox, blonanserin, brexpiprazole, bromperidol, bupropion, busprione, capuride, cariprazine, carpipramine, chlorpromazine, chlorprothixene, clocapramine, clopenthixol, cloperidone, clotiapine, clozapine, cyamemazine, dexclamol, divalproex, dixyrazine, droperidol, flupentixol tiotixene, flupentixol, fluphenazine, fluphenazine, fluspirilene, haloperidol, hydroxyzine, iloperidone, levomepromazine, loxapine, lurasidone, melperone, mesoridazine, molindone, moperone, mosapramine, nefazodone, nemonapride, olanzapine, paliperidone, penfluridol, perazine, pericyazine, perlapine, perospirone, perphenazine, perphenazine, phenelzine, pimavanserin, pimozide, pipamperone, pipotiazine, prochlorperazine, promazine, promethazine, prothipendyl, quetiapine, remoxipride, risperidone, roletamide, sertindole, sulpiride, sultopride, thioproperazine, thioridazine, thiothixene, timiperone, tranylcypromaine, trazodone, trepipam, trifluoperazine, triflupromazine, trimipramine, veralipride, zicronapine, ziprasidone, zotepine, or zuclopenthixol.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, butabarbital, butalbital, capuride, carbocloral, chloral betaine, chloral hydrate, clonazepam, clorazepate, chlordiazepoxide, clorethate, cyprazepam, desipramine, dexclamol, diazepam, dichloralphenazone, divalproex, diphenhydramine, doxepin, estazolam, ethchlorvynol, etomidate, fenobam, flupentixol, fiurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nisobamate, nitrazepam, nortriptyline, oxazepam, paraldehyde, paroxetine, pentobarbital, perlapine, phenelzine, phenobarbital, prazepam, propofol, protriptyline, quazepam, reclazepam, roletamide, secobarbital, sertraline, suproclone, temazepam, tracazolate, tranylcypromaine, trazodone, triazolam, trepipam, tricetamide, triclofos, trimetozine, trimipramine, uldazepam, venlafaxine, zaleplon, zolazepam, or zolpidem.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide; venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

### VI. Formulation and Administration

The presently disclosed subject matter provides a method for administering a one or more compounds formula (I), formula (II), or formula (III) described hereinabove that inhibit Kv11.1-3.1 containing potassium channels to a patient suffering from a condition, or prone to a condition, that is responsive to treatment or prevention with the compound. The method comprises administering, e.g. orally or parenterally, a therapeutically effective amount of the compound, preferably provided as part of a pharmaceutical preparation.

The presently disclosed subject matter also provides pharmaceutical preparations comprising one or more compounds formula (I), formula (II), or formula (III) described hereinabove that inhibit Kv11.1-3.1 containing potassium channels in combination with a pharmaceutical excipient. Modes of administration include administration by injection, e.g. parenteral, intravenous, intraarterial, intramuscular, subcutaneous, and intrathecal, as well as pulmonary, rectal, transdermal, transmucosal, and oral delivery.

The presently disclosed compounds formula (I), formula (II), or formula (III) can be administered in immediate release form, controlled release form or extended release form. Controlled release forms, e.g., sustained, pulsatile, or delayed, are contemplated in the presently disclosed subject matter. In addition to primates, such as humans, a variety of other mammals can be treated according to the presently disclosed methods. For instance, mammals including, but not limited to, cows, sheep, goats, horses, dogs, cats, guinea pigs, or other bovine, ovine, equine, canine, feline, or rodent, such as mouse, species can be treated. However, the method can also be practiced in other species, such as avian species (e.g., chickens).

The presently disclosed compounds may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

Suitable formulation types for parenteral administration include ready-for-injection solutions, dry powders for combination with a solvent prior to use, suspensions ready for injection, dry insoluble compositions for combination with a vehicle prior to use, emulsions and liquid concentrates for dilution prior to administration.

The pharmaceutical carrier(s) employed may be solid or liquid. Liquid carriers can be used in the preparation of solutions, emulsions, suspensions and pressurized compositions. The compounds are dissolved or suspended in a pharmaceutically acceptable liquid excipient. Suitable examples of liquid carriers for parenteral administration include, but are not limited to, water (which may contain additives, e.g., cellulose derivatives, preferably sodium carboxymethyl cellulose solution), phosphate buffered saline solution (PBS), alcohols (including monohydric alcohols and polyhydric alcohols, e.g., glycols) and their derivatives, and oils (e.g., fractionated coconut oil and arachis oil). The liquid carrier can contain other suitable pharmaceutical additives including, but not limited to, the following: solubilizers, suspending agents, emulsifiers, buffers, thickening agents, colors, viscosity regulators, preservatives, stabilizers and osmolarity regulators. Exemplary excipients include, without limitation, carbohydrates, inorganic salts, antimicrobial agents, antioxidants, surfactants, buffers, acids, bases, and combinations thereof. A carbohydrate, such as a sugar, a derivatized sugar, such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient also can include an inorganic salt or buffer including, but not limited to, citric acid, sodium chloride, potassium chloride, sodium sulfate, potassium nitrate, sodium phosphate monobasic, sodium phosphate dibasic, and combinations thereof.

A surfactant may be present as an excipient. Exemplary surfactants include, but are not limited to, polysorbates, such as Tween 20 and Tween 80 and pluronics, such as F68 and F88 (both available from BASF, Mount Olive, N.J.); sorbitan esters; lipids, such as phospholipids, such as lecithin and other phosphatidyl cholines, phosphatidyl ethanolamines (although preferably not in liposomal form), and fatty acids and fatty esters.

Acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

For parenteral administration, the carrier can also be an oily ester, such as ethyl oleate and isopropyl myristate. Sterile carriers are useful in sterile liquid form compositions for parenteral administration. Sterile liquid pharmaceutical compositions, solutions or suspensions can be utilized by, for example, intraperitoneal injection, subcutaneous injection, intravenously, or topically. The compositions can also be administered intravascularly or via a vascular stent.

For pressurized compositions, the liquid carrier can be a halogenated hydrocarbon or other pharmaceutically acceptable propellant. Such pressurized compositions may also be lipid encapsulated for delivery via inhalation. For administration by intranasal or intrabronchial inhalation or insufflation, the compositions may be formulated into an aqueous or partially aqueous solution, which can then be utilized in the form of an aerosol.

The compositions may be administered topically, as a solution, cream, or lotion, by formulation with pharmaceutically acceptable vehicles containing the active compound. The compositions can be in a form suitable for use in transdermal devices.

The presently disclosed compositions may be orally administered, in formulations, such as capsules, tablets, powders or granules, or as suspensions or solutions in water or non-aqueous media. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

The amount of the compound of formula (I), formula (II), or formula (III) in the composition will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the Kv11.1-3.1 containing potassium channel-inhibiting compound in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then further exploring the range at which optimal performance is attained with no significant adverse effects. Generally, however, the excipient will be present in the composition in an amount of about 1% to about 99% by weight, preferably from about 5%-98% by weight, more preferably from about 15-95% by weight of the excipient, with concentrations less than 30% by weight most preferred.

The foregoing pharmaceutical excipients, along with other excipients, are described in “Remington: The Science & Practice of Pharmacy”, 21st ed., Williams & Williams, (2005), the “Physician's Desk Reference”, 67th ed., PDR Network, Montvale, N.J. (2013), and Kibbe, A. H., “Handbook of Pharmaceutical Excipients”, 7th Edition, Pharmaceutical Press, Washington, D.C., 2012.

The dose to be administered, both unit dosage and dosing schedule, will vary depend upon the age, weight, and general condition of the subject, as well as the desired therapeutic effect, the route of administration, and the duration of the treatment. Therapeutically effective amounts are known to those skilled in the art and/or are described in the pertinent reference texts and literature. Generally, dosage levels of between 0.001 to 10 mg/kg of body weight daily are administered to the patient. The dosage range will generally be about 0.5 mg to 1.0 g per patient per day, which may be administered in single or multiple doses. In one embodiment, the dosage range will be about 0.5 mg to 500 mg per patient per day; in another embodiment about 0.5 mg to 200 mg per patient per day; and in yet another embodiment about 5 mg to 50 mg per patient per day. The compounds may be administered on a regimen of 1 to 4 times per day, such as once or twice per day.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Synthetic Procedures

Exemplary compounds were prepared via several general synthetic routes set forth in the Examples below. Notable are Schemes listed below:

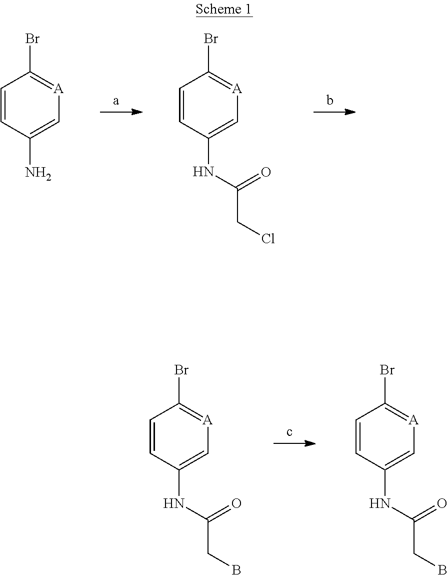

wherein:

A is CH or N;

B is a secondary amine; and

R is a primary or secondary amine;

Step a is a reaction containing chloroacetyl chloride, triethylamine, and CH₂Cl₂, carried out at 0° C. Step b is a reaction containing B, K₂CO₃, and acetonitrile, carried out at 60° C. Step c is a reaction containing R, a catalyst system chosen from either palladium precat G₂ Xantphos, Josiphos with palladium (0) bis(dibenzylideneacetone) or 1,3-bis-(2,6-diisopropylphenyl)-imidazolium with palladium (0) bis(dibenzylideneacetone), Cs₂CO₃ or K₂CO₃, and dimethylsulfoxide carried out at 110° C.

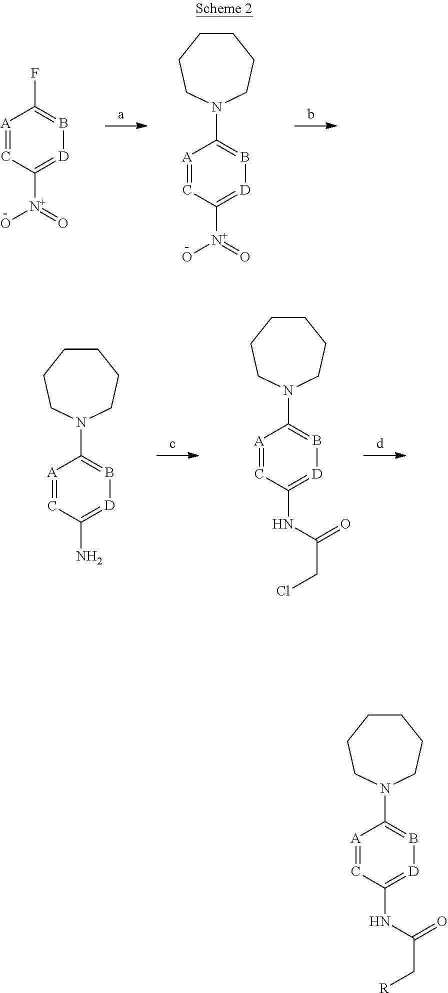

wherein:

A is CH or N;

B is CH or CF;

C is CH or N or CF;

D is CH or N; and

R is a primary or secondary amine.

Step a is a reaction containing azepane, K₂CO₃, and acetonitrile carried out at 60° C. Step b is a reaction containing 10% Pd(C), 1 atm H₂, and ethanol or methanol, or a mixture of both. Step c is a reaction containing chloroacetyl chloride, triethylamine, and CH₂Cl₂, carried out at 0° C. Step d is a reaction containing R and dimethyl formamide carried out at 80° C.

Any of the presently disclosed compounds can be prepared according to one or more of these synthetic routes or specific examples, or via modifications thereof accessible to the person of ordinary skill in the art. Assessment of final compounds was done by one of two analytical methods on an Agilent LC/MS (Agilent Technologies, Santa Clara, Calif.). Method 1: A 7-min gradient of 4% to 100% acetonitrile (containing 0.025% trifluoroacetic acid) in water (containing 0.05% trifluoroacetic acid) was used with an 8-min run time at a flow rate of 1.0 mL/min. Method 2: A 3-min gradient of 4% to 100% acetonitrile (containing 0.05% trifluoroacetic acid) in water (containing 0.05% trifluoroacetic acid) was used with a 4.5-min run time at a flow rate of 1.0 mL/min. A Phenomenex Luna C18 column (3 micron, 3×75 mm) was used at a temperature of 50° C. Purity determination was performed using an Agilent diode array detector for both Method 1 and Method 2. Mass determination was performed using an Agilent 6130 mass spectrometer with electrospray ionization in the positive mode.

### Example 1: N-[4-(1,4-oxazepan-4-yl)phenyl]-2-(1-piperidyl)acetamide hydrochloride

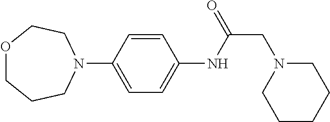 [SMILES: O=C(CN1CCCCC1)Nc1ccc(N2CCCOCC2)cc1]

Example 1 was synthesized in accordance with Scheme 1.

### Step One: N-(4-bromophenyl)-2-chloro-acetamide

To a stirring 0° C. solution of 5 g 4-bromoaniline (29.07 mmol) (2) and 12.09 mL (87.2 mmol) triethylamine in 100 mL CH₂Cl_{2, 2.86} mL (29.07 mmol) chloroacetyl chloride was added drop wise into the solution and allowed to warm to room temperature for 24 hours. The reaction mixture was then diluted with CH₂Cl₂, washed with 1N aqueous hydrochloric acid, washed with brine, and then dried over sodium sulfate. The mixture was filtered, concentrated and purified by flash chromatography (Eiotage lsolera One, 100 g silica gel, linear gradient 0-50% EtOAc:heptanes) to yield 2.92 g of the title compound (40%). ¹H NMR (Chloroform-d, 400 MHz) δ: 8.24 (br s, 1H); 7.47 (d, 4H, J=1.8 Hz); 4.19 (s, 2H). MS [M+1]=249.9.

### Step Two: N-(4-bromophenyl)-2-(1-piperidyl)acetamide

To a stirring solution of 2.85 (11.47 mmol) N-(4-bromophenyl)-2-chloro-acetamide and 3.96 g (28.67 mmol) potassium carbonate in 50 mL CH₃CN, 1.53 g (12.6 mmol) piperidine hydrochloride was added. The reaction mixture was stirred at 80° C. for 6 hours. The solution was cooled to room temperature, filtered and concentrated. The residue was crystallized from ether/heptane to give 3.03 g of the title compound (89%). ¹NNMR (Chloroform-d, 400 MHz) δ: 9.31 (br s, 1H); 7.47 (d, 4H, J=11.7 Hz); 3.06 (s, 2H); 2.56 (br s, 4H); 1.67-1.63 (m, 4H); 1.50 (m, 2H). MS [M+1]=297.

### Step Three: N-[4-(1,4-oxazepan-4-yl)phenyl]-2-(1-piperidyl)acetamide hydrochloride

To a stirring solution of 0.1 g (0.34 mmol) N-(4-bromophenyl)-2-(1-piperidyl)acetamide, 0.053 g (0.5 mmol) 1,4-oxazapane and 0.329 g (1.01 mmol) cesium carbonate in 2 mL DMSO, 0.012 g (0.02 mmol) Xanphos precat G2²⁴⁰ was added. The reaction mixture was heated to 110° C. for 12 hours. The solution was filtered, purified by reverse phase HPLC (C18, linear gradient, 10%-95% CH₃CN/Water, 0.05% TFA additive), concentrated, then treated with 2N HCl in ether and filtered to afford 0.043 g of the title compound (21%). ¹H NMR (DMSO-d6, 400 MHz) δ: 10.81(s, 1H); 7.63 (m, 2H); 7.40 (m, 2H); 4.41 (br s, 4H); 4.13 (d, 2H, J=5 Hz); 3.55-3.45 (m, 4H); 3.11-3.01 (m, 4H); 1.79-1.68 (m, 6H); 1.39 (m, 2H). MS [M+1]=332.2.

### Example 2: 2-(8-azabicyclo[3.2.1]octan-8-yl)-N-16-(3-azabicyclo[3.2.1]octan-3-yl)-3-pyridyljacetamide

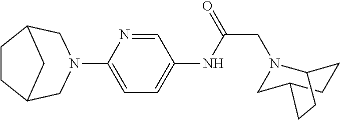 [SMILES: O=C(CN1CC2CCC1CC2)Nc1ccc(N2CC3CCC(C3)C2)nc1]

Prepared analogously to Example 1 according to Scheme 1 using 2-bromo-5-amino-pyridine in Step 1, 8-azabicyclo[3.2.1]octane hydrochloride as the reacting amine in Step 2, and JosiPhos as the palladium ligand in Step 3 to afford the title compound. ¹H NMR (DMSO-d6, 400 MHz) δ: 9.62 (s, 1H); 8.34 (d, 1H, J=2.8 Hz); 7.84 (dd, 1H, J=9.6 Hz & 2.5 Hz); 7.03 (d, 1H, J=8.5 Hz); 4.00-3.95 (m, 4H); 3.80 (d, 2H, J=10.1 Hz); 2.99 (d, 2H, J=11.2 Hz); 2.37 (m, 2H); 2.16-1.93 (m, 6H); 1.68-1.48 (m, 10H). MS [M+1]=355.2.

### Example 3: 2-(8-azabicyclo[3.2.1]octan-8-yl)-N-16-(4-methyl-1-piperidyl)-3-pyridyljacetamide

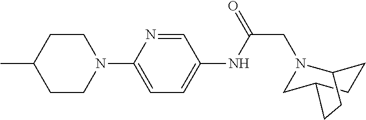 [SMILES: CC1CCN(c2ccc(NC(=O)CN3CC4CCC3CC4)cn2)CC1]

Prepared analogously to Example 2 according to Scheme 1 using 1,3-bis-(2,6-diisopropylphenyl)-imidazolium chloride as the palladium ligand in Step 3 to afford the title compound. ¹H NMR (DMSO-d6, 400 MHz) δ: 9.60 (br s, 1H); 8.32 (d, 1H. J=2.3 Hz); 7,79 (dd, 1H, J=2.5, 9.3 Hz); 7.03 (d, 1H, J=8.6 Hz); 4.19 (d, 2H, J=12.6 Hz); 4.00 (d, 2H, J=21 Hz); 2.85 (m, 2H): 2.16-1.93 (m, 5H); 1.69-1.51 (m, 7H); 1.11 (m, 2H); 0.91 (d, 3H, J=6.3 Hz). MS [M+1]=343.2.

### Example 4: 2-(8-azabicyclo[3.2.1]octan-8-yl)-N-[6-(cyclohexylamino)-3-pyridyl]acetamide

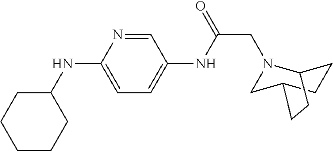 [SMILES: O=C(CN1CC2CCC1CC2)Nc1ccc(NC2CCCCC2)nc1]

Prepared analogously to Example 3 according to Scheme 1 to afford 0.032 g of the title compound. ¹H NMR (DMSO-d6, 400 MHz) δ: 9.86 (s, 1H); 8.30 (s, 1H); 7.81 (d, 1H, J=9.4 Hz); 7.00 (s, 1H); 4.00-3.96 (m, 4H); 3.57 (m, 1H); 2.16-1.92 (m, 8H); 1.72-1.51 (m, 8 H); 1.35-1.11 (m, 5 H). MS [M+1]=343.2.

### Example 5: N-(6-anilino-3-pyridyl)-2-(8-azabicyclo[3.2.1]octan-8-yl)acetamide

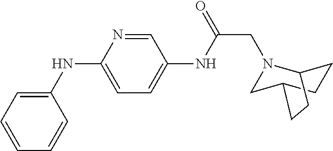 [SMILES: O=C(CN1CC2CCC1CC2)Nc1ccc(Nc2ccccc2)nc1]

Prepared analogously to Example 3 according to Scheme 1 to afford the title compound. ¹H NMR (DMSO-d6, 400 MHz) δ: 9.59 (br s, 1H); 9.10 (br s, 1H); 8.38 (d, 1H. J=2.6 Hz); 7.80 (dd, 1H, J=2.8, 9.1 Hz); 7.65 (m, 2H); 7.26 (m, 2H); 6.90 (m, 2H); 4.01 (br s, 2H); 3.95 (d, 2H, J=5 Hz); 2.17-1.92 (m, 6H); 1.67-1.64 (m, 3H); 1.55 (m, 1H). MS [M+1]=337.2.

### Example 6: N-[6-(3-azabicyclo[3.2.2]nonan-3-yl)-3-pyridyl]-2-(8-azabicyclo[3.2.1]octan-8-yl)acetamide

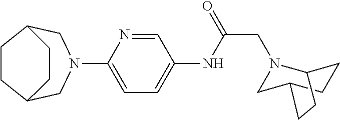 [SMILES: O=C(CN1CC2CCC1CC2)Nc1ccc(N2CC3CCC(CC3)C2)nc1]

Prepared analogously to Example 3 according to Scheme 1 to afford the title compound. ¹H NMR (DMSO-d6, 400 MHz) δ: 9.63 (br s, 1H); 8.31 (d, 1H. J=2.5 Hz); 7.81 (dd, 1H, J=2.3, 9.6 Hz): 7.11 (d, 1H, J=8.9 Hz); 4.00 (br s, 2H); 3.96 (d, 2H, J=5.5 Hz); 3.71 (d, 2H, J=4.1 Hz); 2.16-1.91 (m, 8H); 1.66-1.51 (m, 12H). MS [M+1]=369.2.

### Example 7: N-[4-(azepan-1-yl)phenyl]-2-(1,4-oxazepan-4-yl)acetamide hydrochloride

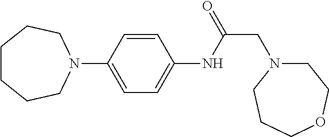 [SMILES: O=C(CN1CCCOCC1)Nc1ccc(N2CCCCCC2)cc1]

Example 7 was synthesized in accordance with Scheme 2.

### Step One: 1-(4-nitrophenyl)azepane

To a stirring solution of 5 g (35.44 mmol) 1-fluro-4-nitrobenzene and 4.39 mL (38.98 mmol) azepane in 50 mL CH₃CN, 12.24 g (88.59 mmol) potassium carbonate was added. The reaction mixture was stirred at 70° C. for three hours. The solution was diluted with ethyl acetate, washed with water, dried with MgSO₄, filtered, concentrated and purified by flash chromatography (Biotage Isolera One, 1.00 g silica gel, linear gradient 0-10% MeOH:CH₂CL₂) to afford 7.6 g of the title compound (97%). ¹H NMR (Chloroform-d, 400 MHz) δ: 8.11(d, 2H, J=9.4 Hz); 6.63 (d, 2H, J=9.3 Hz); 3.57 (t, 4H, J=6.1 Hz); 1.85 (m, 4H); 1.59 (m, 4H). MS [M+1]=221.1.

### Step Two: 4-(azepan-1-yl)aniline

To a solution of 7.6 g (34.5 mmol) 1-(4-nitrophenyl)azepane in 100 mL ethanol, 2 g (34.5 mmol) 10% palladium on carbon was added. The solution was stirred under 1 atm hydrogen gas for 24 hours. The solution was filtered and concentrated to give 6.4 g of the title compound (97%). ¹H NMR (Chloroform-d, 400 MHz) δ: 6.67-6.56 (m, 4H); 3.4 (m, 4H); 3.22 (m, 2H); 1.78 (br s, 4H); 1.46-1.68 (m, 4H). MS [M+1]=190.1.

### Step Three: N-[4-(azepan-1-yl)phenyl]-2-chloro-acetamide

To a solution of 0.5 g (2.62 mmol) 4-(azepan-1-yl)aniline in 13 mL CH₂Cl₂ was added 330 μL (2.4 mmol) triethylamine. The reaction mixture was stirred for 1 minute then to which was added 251 μL (3.2 mmol) chioroacetyl chloride. The reaction mixture was stirred and allowed to warm to room temperature for two hours and then diluted with 20 mL CH₂Cl₂, washed with water, dried over MgSO₄, filtered, and concentrated. Purification by flash chromatography (Biotage Isolera One, 24 g silica gel, linear gradient 20%-60% EtOAc:heptanes) afforded 0.65 g of the title compound (93%), 1H NMR (DMSO-d6, 400 MHz) 9.97 (s, 1H); 7.37 (d, 2H, J=8.6 Hz); 6.66 (br s, 2H); 4.18 (s, 2H); 3.44 t, 4H, J=5.8 Hz); 1.72 (m, 4H); 1.46 (m, 4H). MS [M+1]=267.1.

### Step Four (General procedure A): N-[4-(azepan-1-yl)phenyl]-2-(1,4-oxazepan-4-yl)acetamide hydrochloride

To a stirring solution of 0.030 g (0.112 mmol) N-[4-(azepan-1-yl)phenyl]-2-chloro-acetamide in 1 mL DMF was added 0.036 g (0.336 mmol) 1,4-oxazepane, which was then heated to 80° C. for 3 hours. The reaction mixture was then diluted with 0.5 mL MeOH and purified by reverse phase HPLC (C18, linear gradient 10%-95% ACN/Water, 0.05% TFA additive) yielding 0.04 g (30%) of the title compound. ¹H NMR (DMSO-d6, 400 MHz) δ: 10.34 (br s, 1H); 7.52 (br s, 2H); 6.79 (br s, 2H); 4.22 (s, 2H); 3.90- 3.69 (m, 4H); 3.53-3.39 (m, 8H); 2.25-1.55 (m, 10H). MS [M+1]=332.2.

Resulting compounds using General Procedure A were carried out using various primary or secondary amines and were tested as 1:1 TFA salts, unless noted otherwise by different ratios or as the hydrochloride salt.

### Example 8: N-[4-(azepan-1-yl)phenyl]-2-(3-fluoro-1-piperidyl)acettamide

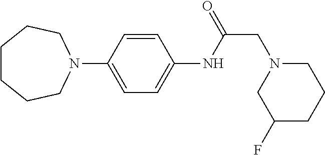 [SMILES: O=C(CN1CCCC(F)C1)Nc1ccc(N2CCCCCC2)cc1]

Prepared according to General Procedure A to afford the title compound. ¹H NMR (DMSO-d6, 400 MHz) δ: 10.23 (s. 1H); 7.36 (d, 2H, J=9.1 Hz); 6.67 (d, 2H, J=8.8 Hz); 5.15 (m, 1H); 4.13 (m, 2H); 3.8 (m, 1H); 3.43 (m, 5H); 3.22 (br s, 1H); 2.49 (m, IH); 2.07-1,91 (m, 2H); 1.77-1.70 (m, 6H); 1.47-1.44 (m, 4H). MS [M+1]=334.2.

### Example 9: N-[4-(azepan-1-yl)pheny]-2-[2-(trifluoromethyl)-1-piperidyl]acetamide

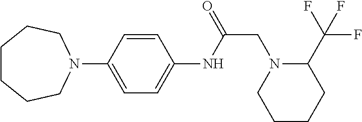 [SMILES: O=C(CN1CCCCC1C(F)(F)F)Nc1ccc(N2CCCCCC2)cc1]

Prepared according to General Procedure A to afford the title compound. ¹H NMR (DMSO-d6, 400 MHz) δ: 9.45 (s, 1H); 7.41 (d, 2H, J===7.8 Hz); 6.76 (br s, 2H), 3.70 (m, 1H); 3.46-3.39 (m, 5H); 2.89-2.73 (m, 2H), 1.74-146 (m, 14H). MS M+1]=384.2.

### Example 10: N-[4-(azepan-1-yl)phenyl]-2-[4-(hydroxymethyl)-1-piperidyl]acetamide

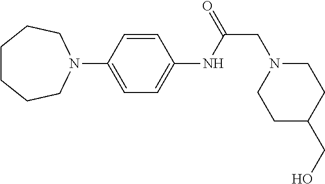 [SMILES: O=C(CN1CCC(CO)CC1)Nc1ccc(N2CCCCCC2)cc1]

Prepared according to General Procedure A to afford the title compound. ¹H NMR (DMSO-d6, 400 MHz) δ: 10.25 (s, 1H) 7.36 (d, 2H, J=9.1 Hz); 6.67 (d, 2H, J=9 Hz); 4.02 (m, 2H); 3.53 (m, 2H); 3.43 (t, 4H, J=5.9 Hz); 3.27 (m, 3H); 3.05 (m, 2H); 1.84 (m, 2H); 1.70 (br s, 4H); 1.61 (m, 2H): 1.51-1.43 (m, 5H). MS [M+1]=346.3.

### Example 11: N-[4-(azepan-1-yl)phenyl]-2-[2-(hydroxymethyl)-1-piperidyl]acetamide

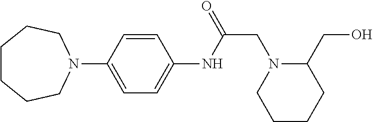 [SMILES: O=C(CN1CCCCC1CO)Nc1ccc(N2CCCCCC2)cc1]

Prepared according to General Procedure A to afford the title compound. ¹H NMR (DMSO-d6, 400 MHz) δ: 10.29 (s, 1H); 7.43 (m, 5H), 7.27 (d, 1H, J=7.3), 6.67 (d. 1H, J=8.8 Hz) 4.07 (s, 2H); 3.55 (m, 2H); 3.44 (t, 5H, J=5.8 Hz); 3.32-3.17 (m, 2H); 1.94 (m, 4H); 1.70 (m, 5H); 1.45 (m, 4H). MS [M+1]=346.3.

### Example 12: N-[4-(azepan-1-yl)phenyl]-2-[4-(1-hydroxy-1-methyl-ethyl)-1-piperidyl]acetamide

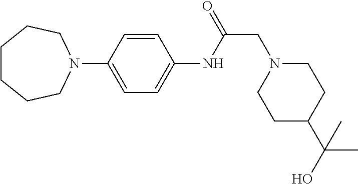 [SMILES: CC(C)(O)C1CCN(CC(=O)Nc2ccc(N3CCCCCC3)cc2)CC1]

Prepared according to General Procedure A to afford the title compound. ¹H NMR (DMSO-d6, 400 MHz) δ: 10.23 (s, 1H); 7.35 (d, 2H, J=9.1 Hz); 6.67 (d, 2H, J=8.8 Hz); 4.00 (d, 2H, J=5.1 Hz); 3.54 (m, 3H); 3.43 (t, 4H, J=5.8 Hz); 3.30 (m, 2H); 1.85 (m, 4H); 1.70-1.60 (m, 7H); 1.44-1.36 (m, 8H). MS [M+1]=374.3.

### Example 13: N-[4-(azepan-1-yl)phenyl]-2-[4-(4-chlorophenyl)-4-hydroxy-1-piperidyl]acetamide

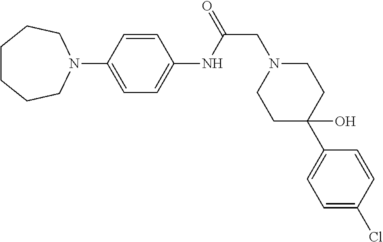 [SMILES: O=C(CN1CCC(O)(c2ccc(Cl)cc2)CC1)Nc1ccc(N2CCCCCC2)cc1]

Prepared according to General Procedure A to afford the title compound. ¹H NMR (DMSO-d6, 400 MHz) δ: 10.26 (s, 1H); 7.50 (m, 4H); 7.38 (d, 2H, J=9.1 Hz); 6.68 (d, 2H, J=9.1 Hz); 5.63 (br s, 1H); 4.16 (br d, 2H, J=4.6 Hz); 3.46 (m, 8H); 2.33 (m, 2H); 1.82 (br d, 2H, J=13.9 Hz); 1.71 (br s, 4H); 1.45 (m, 4H). MS [M+1]=442.2.

### Example 14: N-[4-(azepan-1-yl)phenyl]-2-pyrrolidin-1-yl-acetamide hydrochloride

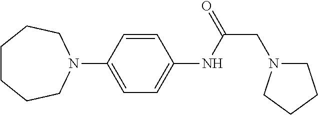 [SMILES: O=C(CN1CCCC1)Nc1ccc(N2CCCCCC2)cc1]

Prepared according to General Procedure A to afford the title compound. ¹H NMR (DMSO-d6, 400 MHz) δ: 10.24 (br s, 1H); 7.47 (br s, 2H); 6.76 (br s, 2H); 4.20 (d, 2H, J=4.0 Hz); 3.60 (d, 2H, J=5.6 Hz); 3.46 (m, 4H); 3.12 (d, 2H, J=10.9 Hz); 2.01-1.45 (m, 12H). MS [M+1]=302.2.

### Example 15: N-[4-(azepan-1-yl)phenyl]-2-(3-methyl-1-piperidypacetamide

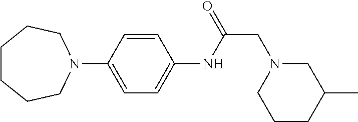 [SMILES: CC1CCCN(CC(=O)Nc2ccc(N3CCCCCC3)cc2)C1]

Prepared according to General Procedure A to afford the title compound. ¹H NMR (DMSO-d6, 400 MHz) δ: 10.24 (s, 1H); 7.37 (d, 2H, J=9.1); 6.68 (d, 2H, J=9.1); 4.02 (m, 2H); 3.43 (m: 6H); 2.94 (m, 1H); 2.70 (m: 1H); 1.96 (m, 1H); 1.80-1.71 (m, 7H); 1.46 (m: 4H); 0.90 (m, 4H). MS [M+1]=330.3.

### Example 16: N-[4-(azepan-1-yl)phenyl]-2-[2-(4-chlorophenyl)-1-piperidyl]acetamide

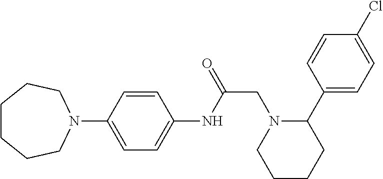 [SMILES: O=C(CN1CCCCC1c1ccc(Cl)cc1)Nc1ccc(N2CCCCCC2)cc1]

Prepared according to General Procedure A to afford the title compound. ¹H NMR (DMSO-d6, 400 MHz) δ: 10.00 (s, 1H); 7.61-7.54 (m, 4H); 7.23 (d, 2H, J=9.1 Hz); 6.63 (d, 2H, J=9.1 Hz); 4.54 (br d, 1H, J=11.1 Hz); 3.74-3.63 (m, 3H); 3.42 (t, 4H, J=5.9 Hz); 3.28 (m, 1H); 2.18 (m, 1H); 2.04 (m, 1H) 1.90 (m, 3H); 1.69-1.6 (m, 5H); 1.44 (m, 4H), MS [M+1]=426.2.

### Example 17: N-[4-(azepan-1-yl)phenyl]-2-[3-(3-chlorophenyl)-1-piperidyl]acetamide

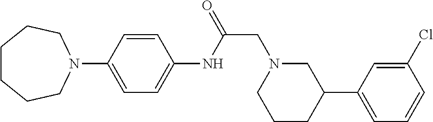 [SMILES: O=C(CN1CCCC(c2cccc(Cl)c2)C1)Nc1ccc(N2CCCCCC2)cc1]

Prepared according to General Procedure A to afford the title compound. ¹H NMR (DMSO-d6, 400 MHz) δ: ¹H NMR (DMSO-d6, 400 MHz) δ10.29 (s, 1H), 7.43 (m, 5H): 7.27 (d, 1H, J=7.3): 6.67 (d, 1H, J=8.8 Hz) 4.07 (s, 2H); 3.55 (m, 2H); 3.44 (t: 5H, J=5.8 Hz); 3.32-3.17 (m, 2H); 1.94 (m, 4H): 1.70 (m, 5H); 1.45 (m, 4H). MS [M+1]=426.2.

### Example 18: N-[4-(azepan-1-yl)phenyl]-2-[4-(3-cyanophenyl)-1-piperidyl]acetamide

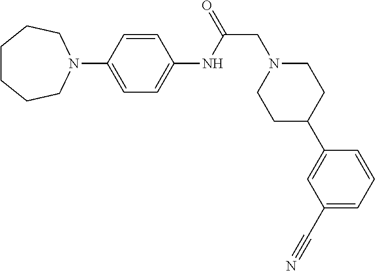 [SMILES: N#Cc1cccc(C2CCN(CC(=O)Nc3ccc(N4CCCCCC4)cc3)CC2)c1]

Prepared according to General Procedure A to afford the title compound. ¹H NMR (DMSO-d6, 400 MHz) δ: 10.28 (s, 1H); 7.81 (m, 2H); 7.66 (m, 2H); 7.36 (d, 2H, J=7.3 Hz); 6.67 (d, 2H, J=7.6 Hz); 4.06 (m, 2H); 3.56 (m, 2H); 3.42-3.25 (m, 6H); 3.06 (br s, 1 H); 1.94 (m, 3H); 1.70 (br s, 5H); 1.44 (m, 4H). MS [M+1]=417.2.

### Example 19: N-[4-(azepan-1-yl)phenyl]-2-(2-methyl-4-phenyl-1-piperidyl)acetamide

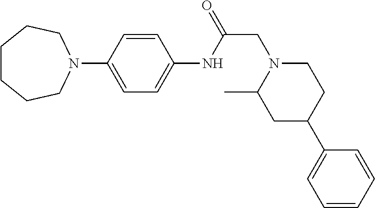 [SMILES: CC1CC(c2ccccc2)CCN1CC(=O)Nc1ccc(N2CCCCCC2)cc1]

Prepared according to General Procedure A to afford the title compound as a mixture of isomers. Data for major isomer: ¹H NMR (DMSO-d6. 400 MHz) δ: 10.30 (s, 1H); 7.39-7.24 (m, 7 H); 6.69 (s, 2H, J=8.9 Hz); 4.37 (d, 2H, J=5.4 Hz); 3.67-3.64 (m, 2H); 3.44 (t, 4H, J=6 Hz); 3.31 (m, 1H); 2.91 (m, 1H); 2.03-1.97 (m, 4H); 1.71 (m, 4H); 1.45 (m, 4H); 1.34 (d, 3H, J=6.4 Hz). MS [M+1]=406.2.

### Example 20: N-[4-(azepan-1-yl)phenyl]-2-[3-(4-fluorophenyl)pyrrolidin-1-yl]acetamide

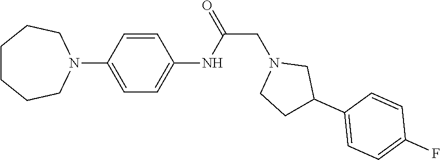 [SMILES: O=C(CN1CCC(c2ccc(F)cc2)C1)Nc1ccc(N2CCCCCC2)cc1]

Prepared according to General Procedure A to afford the title compound. ¹H NMR (DMSO-d6, 400 MHz) δ: 10.08 (s; 1H); 7.60 (m, 1H); 7.51 (m, 3H); 7.23 (d, 2H, J=8.1 Hz), 6.63 (d, 2H, J=9.1 Hz); 4.57 (m, 1H), 4.01 (m, 2H); 3.82 (d, 1H, J=11.4); 3.42 (m, 5H); 2.40 (m, 1H); 2.18 (m, 3H); 1.69 (br s, 4H); 1.45 (m, 4H). MS [M+1]=396.2.

### Example 21: 2-(8-azabicyclo[3.2.1]octan-8-yl)-N-[4-(azepan-1-yl)phenyl]acetamide

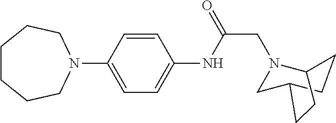 [SMILES: O=C(CN1CC2CCC1CC2)Nc1ccc(N2CCCCCC2)cc1]

Prepared according to General Procedure A to afford the title compound. ¹H NMR (DMSO-d6, 400 MHz) δ: 10.14 (s. 1H); 7.35 (d, 2H, J=8.9 Hz); 6.67 (d, 2H, J=9.1 Hz); 3.97 (by s, 2H); 3.87 (d, 2H, J=5.5 Hz); 3.43 (t, 4H, J=6.1), 2.15 (m, 2H); 2.03 (m, 1H); 1.93 (m, 2H); 1.70 (m, 7H); 1.53 (m, 2H); 1.45 (m, 4H). MS [M+1]=342.2.

### Example 22: N-[4-(azepan-1-yl)phenyl]-2-(3-benzyloxy-1-piperidyl)acetamide

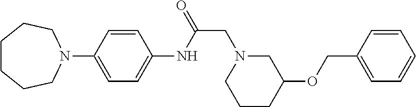 [SMILES: O=C(CN1CCCC(OCc2ccccc2)C1)Nc1ccc(N2CCCCCC2)cc1]

Prepared according to General Procedure A to afford the title compound. ¹H NMR (DIVISO-d6, 400 MHz) δ: 10.22 (s, 1H); 7.44-7.31 (m, 7H); 6.68 (d, 2H, J=9.1 Hz); 4.67-4.53 (m, 2 H); 4.13-3.99 (m, 2H); 3.90-3.64 (m, 2H); 3.44-3.25 (m, 6 H); 3.01 (br s, 1H); 2.07-1.92 (m, 2H); 1.71 (m, 5H); 1.46 (m, 5H). MS [M+1]=422.2.

### Example 23: N-[4-(azepan-1-yl)phenyl]-3-(1-piperidyl)propenamide

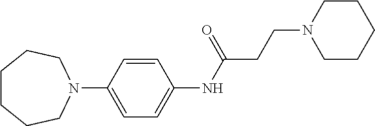 [SMILES: O=C(CCN1CCCCC1)Nc1ccc(N2CCCCCC2)cc1]

### Step One: N-[4-(azepan-1-yl)phenyl]-3-chloro-propanamide

To a 4° C. solution of 0.03 g (0.158 mmol) 4-(azepan-1-yl)aniline in 1 mL CH₂Cl₂ was added 20 μL (0.1435 mmol) triethylamine. The reaction mixture was stirred for 1 minute then to which was added 23 μL, (0.230 mmol) 3-chloropropionyl chloride. The reaction mixture was stirred and allowed to warm to room temperature for two hours and then diluted with 20 mL CH₂Cl₂, washed with water, dried over MgSO₄, filtered, and concentrated which afforded 0.040 g of mixture containing the title compound which was carried on to the next step. MS [M+1]=281.2.

### Step Two: N-[4-(azepan-1-yl)phenyl]-3-(1-piperidyl)propanamide

Prepared analogously to General Procedure A to afford the title compound. ¹H NMR (DMSO-d6, 400 MHz) δ: 9.84 (s, 1H); 7.36 (d, 2H, J=8.8 Hz); 6.66 (br d, 2H, J=8.9 Hz); 3.45 (m, 6H); 3.34 (m, 2H); 2.92 (m, 2H); 2.77 (br t, 2H, J=7.2 Hz); 1.84(m, 2H); 1.71-1.58 (m, 8H); 1.45 (m, 4H). MS [M+1]=334.2.

### Example 24: N-[4-(azepan-1-yl)phenyl]-2-(1-piperidyl)propanamide

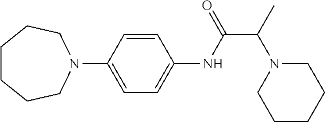 [SMILES: CC(C(=O)Nc1ccc(N2CCCCCC2)cc1)N1CCCCC1]

### Step One: N-[4-(azepan-1-yl)phenyl]-2-chloro-propanamide

To a 4° C. solution of 0.03 g (0.158 mmol) (11) in 1 mL CH₂Cl₂ was added 20 μL (0.1435 mmol) triethylamine. The reaction mixture was stirred for 1 minute then to which was added 23 μL (0.230 mmol) 3-chloropropionyl chloride. The reaction mixture was stirred and allowed to warm to room temperature for two hours and then diluted with 20 mL CH₂CL₂, washed with water, dried over MgSO₄, filtered, and concentrated in vacuo which afforded 0.040 g of mixture containing the title compound. MS [M+1]=281.2.

### Step Two: N-[4-(azepan-1-yl)phenyl]-2-(1-piperidyl)propanamide

Prepared analogously to General Procedure A to afford the title compound. NMR (HMSO-d6, 400 MHz) δ: 10.36 (s, 1H); 7.40 (d, 2H, J=9.1 Hz); 6.70 (d, 2H, J=9.1 Hz); 3.98 (t, 1H, J=6.9 Hz); 3.55 (m, 1H); 3.45-3.35 (m, 5H); 3.07-2.89 (m, 2H); 1.80-1.71 (m, 9H); 1.53-1.45 (m, 8H) MS [M+1]=334.2.

### Example 25: N-[6-(azepan-1-yl)-3-pyridyl]-2-(1-piperidyl)acetamide

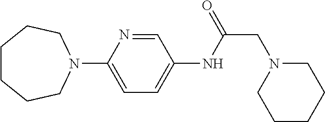 [SMILES: O=C(CN1CCCCC1)Nc1ccc(N2CCCCCC2)nc1]

Prepared analogously to Example 7 according to Scheme 2 using 2-fluro-5-nitro-pyridine in Step 1 to afford the title compound. ¹H NMR (DMSO-d6, 400 MHz) δ: 10.83 (s, HA); 8.3:5 (br d, 1H J=2.5 Hz); 7.86 (dd, 1H, J=2.2, 9.6 Hz); 7.10 (br d, 1H, J-8.9 Hz); 4.11 (s, 2H); 3.65 (t, 4H, J=5.9 Hz); 3.48 (m, 2H); 3.05 (br s, 2H); 1.78 (m, 10 H); 1.50 (m, 4H), MS [M+1]=317.2.

### Example 26: 2-(7-azabicyclo[2.2.1]heptan-7-yl)-N-[6-(azepan-1-yl)-3-pyridyl]acetamide

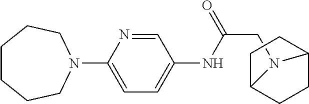 [SMILES: O=C(CN1C2CCC1CC2)Nc1ccc(N2CCCCCC2)nc1]

Prepared analogously to Example 25 according to Scheme 2 to afford the title compound. ¹H NMR (DMSO-d6, 400 MHz) δ: 10.01 (br s, 1H); 8.36 (d, 1H, J=2.5 Hz); 7.88 (dd, 1J=2.2, 9,6 Hz); 7.13 (d, 1H, J=8.6 Hz); 4.20 (br s, 2H); 4.07 (d, 2H, J=6 Hz); 3.66 (t, 4H, J=6.1 Hz); 2.02-2.00 (m, 4H); 1.74 (m, 7H); 1.50 (m, 4H). MS [M+1]=329.2.

### Example 27: N-[6-(azepan-1-yl)-3-pyridyl]-2-[cyclohexylamino]acetamide

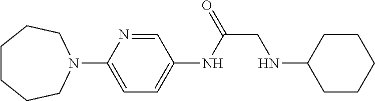 [SMILES: O=C(CNC1CCCCC1)Nc1ccc(N2CCCCCC2)nc1]

Prepared analogously to Example 25 according to Scheme 2 to afford the title compound. ¹H NMR (DMSO-d6, 400 MHz) δ: 8.91 (br s, 2H); 8.31 (d, 1H, J=2.5 Hz); 7.79 (d, 1H, J=7.3 Hz); 6.97 (s, 1H); 3.95 (t, 2H, J=5.8 Hz); 3.63 (t, 4H, J=6 Hz); 3.06 (br s, 1H); 2.02-1.99 (m, 2H); 1.78-1.73 (m, 5H); 1.62-1.59 (m, 1H); 1.48 (m, 4H); 1.32-1.06 (m, 5H). MS [M+1]=331.2.

### Example 28: N-[6-(azepan-1-yl)-3-pyridyl]-2-[cyclohexyhmethyl)amino] acetamide

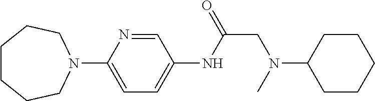 [SMILES: CN(CC(=O)Nc1ccc(N2CCCCCC2)nc1)C1CCCCC1]

Prepared analogously to Example 25 according to Scheme 2 to afford the title compound. ¹H NMR (DMSO-d6, 400 MHz) δ: 9.62 (br s, 1H); 8.34 (d, 1H, J=2.5 Hz); 7.85 (dd, 1H/=2.3, 9,6 Hz); 7.03 (d, 1H, J=7.5 Hz); 4.26 (d, 1H, J=5.4 Hz); 4.01 (m, 1H); 3.65 (t, 4H, J=5.8 Hz); 3.28 (m, 1H); 2.81 (br s, 3H); 2.08-1.95 (m, 2H); 1.84-1.81 (m, 2H); 1.74 (br s, 4H); 1.63-1.59 (m, 1H); 1.50-1.08 (m, 8H). MS [M+1]=345.2.

### Example 29: N-[6-(azepan-1-yl)-3-pyridyl]-2-(cycloheptylamino)acetamide

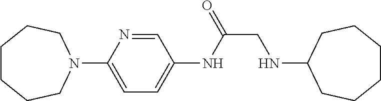 [SMILES: O=C(CNC1CCCCCC1)Nc1ccc(N2CCCCCC2)nc1]

Prepared analogously to Example 25 according to Scheme 2 to afford the title compound. ¹H NMR (DMSO-d6, 400 MHz) δ: 8.84 (br s, 2H); 8.30 (d, 1H, J=2.5 Hz); 7.78 (d, 1H, J=9.4 Hz): 6.93 (s: 1H); 3.94 (t, 2H, J=6.1 Hz); 3.63 (t, 4H, J=6 Hz); 3.26 (br s, 1H); 2.03-4.98 (m, 2H); 1.73 (m, 6H); 1.62-1.39 (m, 12H). MS [M+1]=345.2.

### Example 30: 2-anilino-N-[6-(azepan-1-yl)-3-pyridyl]acetamide

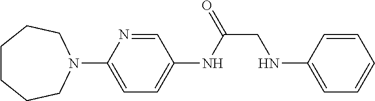 [SMILES: O=C(CNc1ccccc1)Nc1ccc(N2CCCCCC2)nc1]

Prepared analogously to Example 25 according to Scheme 2 to afford the title compound. ¹H NMR (DMSO-d6, 400 MHz) δ: 10.25 (s, 1H); 8.42 (s, 1H); 8.00 (d, 1H, J=9.2 Hz); 7.25-7.05 (m, 5H); 6.62-6.57 (m, 2H); 3.86 (s, 2H); 3.65 (t, 4H, J=6.1 Hz); 1.73 (m, 4H); 1.50 (m, 4H), MS [M+1]=333.2.

### Example 31: N-[6-(azepan-1-yl)-3-pyridyl]-2-(benzylamino)acetamide

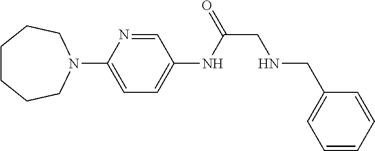 [SMILES: O=C(CNCc1ccccc1)Nc1ccc(N2CCCCCC2)nc1]

Prepared analogously to Example 25 according to Scheme 2 to afford the title compound. ¹H NMR (DMSO-d6, 400 MHz) δ: 9.42 (br s, 2H); 8.28 (d, 1H, J=2.5 Hz); 7.78 (dd, 1H J=2.6, 11.9 Hz); 7.52-7.43 (m, 5H); 6.97 (s, 1H); 4.23 (br s, 2H); 3.87 (br s. 2H); 3.63 (t, 4H, J=6 Hz); 1.72 (m, 4H); 1.48 (m, 4H). MS [M+1]=339.2.

### Example 32: 2-(3-azabicyclo[3.2.1]octan-3-yl)-N-[6-(azepan-1-yl)-3-pyridyl]acetamide

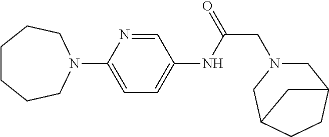 [SMILES: O=C(CN1CC2CCC(C2)C1)Nc1ccc(N2CCCCCC2)nc1]

Prepared analogously to Example 25 according to Scheme 2 to afford the title compound. ¹H NMR (DMSO-d6, 400 MHz) δ: 9.02 (br s, 1H); 8.25 (s, 1H); 7.77 (d, 1H J=8.1 Hz); 6.90 (s, 1H); 4.00 s, 2H); 3.62 (t, 4H, J=6 Hz); 3.45 (m, 2H); 3.19 (m, 2H); 2.38 (m, 3H); 1.83-1.66 (m, 8H); 1.48 (m, 5H). MS [M+1]=343.2.

### Example 33: 2-(8-azabicyclo[3.2.1.]octan-8-yl)-N-[6-(azepan-1-yl)-3- pyridyl]acetamide

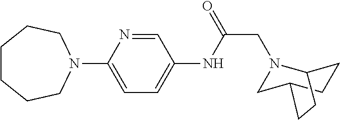 [SMILES: O=C(CN1CC2CCC1CC2)Nc1ccc(N2CCCCCC2)nc1]

Prepared analogously to Example 25 according to Scheme 2 to afford the title compound. ¹H NMR (DMSO-d6, 400 MHz) δ: 10.74 (s, 1H); 8.34 (d, 1H, J=2.3 Hz); 7.86 (m, 1H); 7.04 (m, 1H); 4.00 (m, 4H); 3.65 (t, 4H, J=5.9 Hz); 2.17 (m, 2H); 2.04 (m, 1H); 1.94 (m, 2H); 1.74 (m, 5H); 1.66 (m, 4H); 1,54 (m, 4H). MS [M+1]=343.2.

### Example 34: N[6-(azepan-1-yl)-5-pyridyl]-2-(1-pipendyl)acetamtide

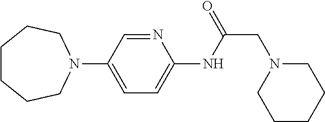 [SMILES: O=C(CN1CCCCC1)Nc1ccc(N2CCCCCC2)cn1]

Prepared analogously to Example 7 according to Scheme 2 using 5-fluro-2-nitro-pyridine in Step 1 to afford the title compound. ¹H NMR (DMSO-d6, 400 MHz) δ: 10.75 (s, 1H); 7.84 (m, 2H); 7.18 (dd, 1H, J=3.4, 9.1 Hz) 4.08 (d, 2H, J=5.6 Hz) 3.47 (m, 6H); 3.01 (m, 2H); 1.77 (m, 10H); 1.45-1.29 (4H). MS [M+1]=317.3.

### Example 35: N-[2-(azepan-1-yl)pyrimidin-5-yl]-2-(1-piperidyl)acetamide

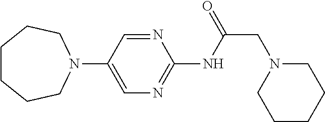 [SMILES: O=C(CN1CCCCC1)Nc1ncc(N2CCCCCC2)cn1]

Prepared analogously to Example 7 according to Scheme 2 using 2-bromo-5-nitro-pyrimidine in Step 1 to afford the title compound. ¹H NMR (DMSO-d6, 400 MHz) δ: 10.44 (s, 1H); 8.48 (s, 2H); 4.08 (d, 2H, J=4.8); 3.71 (t, 4H, J=5.9); 3.48 (m, 2H); 3.02 (m, 2H); 1.78 (m, 10H); 1.49 (m, 4H). MS [M+1]=318.2.

### Example 36: N-[4-(azepan-1-yl)-3-fluoro-phenyl]-2-(1-piperidyl)acetamide

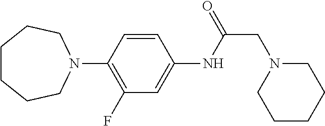 [SMILES: O=C(CN1CCCCC1)Nc1ccc(N2CCCCCC2)c(F)c1]

Prepared analogously to Example 7 according to Scheme 2 using 3,4-difluronitrobenzene in Step 1 to afford the title compound. ¹H NMR (DMSO-d6, 400 MHz) δ: 10.48 (s, 1H); 7.46 (dd, 1H, J=2.6, 16 Hz); 7.14 (dd, 1H, J=2.5,8.8 Hz); 6.93 (m, 1H); 4.05 (d, 2H, J=4.8 Hz); 3.47 (m, 2H); 3.30 (t, 4H, J=5.7); 3.02 (m, 2H); 1.77 (m, 10H); 1.55 (m, 4H). MS [M+1]=334.2.

### Example 37: 2-(8-azabicyclo[3.2.1]octan-8-yl)-N-[4-(azepan-1-yl)-3-fluoro-phenyl]acetamide

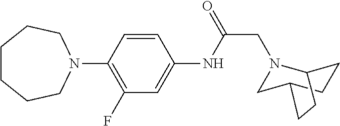 [SMILES: O=C(CN1CC2CCC1CC2)Nc1ccc(N2CCCCCC2)c(F)c1]

Prepared analogously to Example 36 according to Scheme 2 to afford the title compound. ¹H NMR (DMSO-d6, 400 MHz) δ: 9.55 (br s, 1H); 7.49 (dd, 1H, J=2.5, 15.9 Hz); 7.14 (dd, 1H, J=2.3, 8.8 Hz); 6.93 (m, 1H); 3.99 (m, 2H); 3.91 (d, 2H, 5.8 Hz); 3.28 (t, 4H, J=5.8 Hz); 2.15-2.01 (m, 4H); 1.93 (m, 2H); 1.74-1.63 (m, 7H); 1.54 (m, 5H). MS [M+1]=360.2.

### Example 38: N- 4-(azepan-1-yl)-2-fluoro-phenyl1-2-(1-piperidyl)acetamide

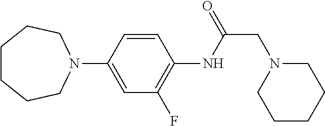 [SMILES: O=C(CN1CCCCC1)Nc1ccc(N2CCCCCC2)cc1F]

Prepared analogously to Example 7 according to Scheme 2 using 2,4-difluoro-1-nitrobenzene in Step 1 to afford the title compound. ¹H NMR (DMSO-d6, 400 MHz) δ: 9.73 (s, 1H); 7.39 (dd, 1H, J=6.2, 8.5 Hz); 6.90 (dd, 1H, J=2.7, 11.6 Hz); 6.76 (td, 1H, J=2.8, 7.9 Hz); 4.12 (s, 2H); 3.48 (d, 2H, J=11.4 Hz); 3.12-3.04 (m, 6H); 1.78-1.62 (14H) MS [M+1]=334.2.

### Example 39: 2-(8-azabicyclo[3.2.1]octan-8-yl)-N-[6-(azepan-1-yl)-5-fluoro-3-pyridyl]acetamide

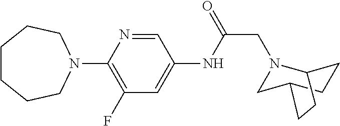 [SMILES: O=C(CN1CC2CCC1CC2)Nc1cnc(N2CCCCCC2)c(F)c1]

Prepared analogously to Example 7 according to Scheme 2 using 2,3-difluoro-5-nitropyridine in Step 1 to afford the title compound. ¹H NMR (DMSO-d6, 400 MHz) δ: 9.62 (br s, 1H); 8.08 (t, 1HT=1.6 Hz); 7.75 (dd, 1H, J=2, 15.6 Hz); 3.99 (m, 2H); 3.94 (d, 2H, J=5.8 Hz); 3.60 (t, 4H, J=5.1 Hz); 2.16-2.13(m, 2H); 2.07-2.00 (m, 2H); 1.94-1.91 (m, 2H); 1.72-1.63 (m, 7H); 1.49 (m, 5H). MS [M+1]=361.2.

### KCNH2-3.1 Inhibition Assay Procedure

The ability of compounds to inhibit the activity of KCNH2-3.1 was determined by an ion flux assay as described (Titus et al, 2009, which is incorporated herein by reference). U2OS cells were cultured in media containing DMEM, 10% FBS, and Penicillin/Streptomycin. Cells were grown to 90% confluency then washed with PBS and pelleted. Cells were resuspended in DMEM containing 2% FBS and incubated with hERG or hERG 3.1 Bacmam virus for 4 hours at room temperature in the dark. Cells were plated onto clear-bottom PDL coated 1536 well plates and allowed to grow 16-20 hours at 37° C. FluxOr loading buffer containing 10mM Red Dye #40 was added to each well and incubated for 45 minutes. Plates were loaded and analyzed in Hamamatsu FDSS (7000) using a two-addition protocol. In the first addition step of the assay, compounds to be tested and the cell compound mixture were incubated together for 5 minutes before addition of FluxOr stimulus buffer as the second addition step. Channel activity was detected and analyzed via the slope of fluorescence intensities versus the time of first 30 s after compound addition was calculated from the kinetic results. The signal-to-basal ratio was calculated as the slope of DMSO solvent controls in the stimulated group divided by the slope of an unstimulated group. The concentration responses of compound inhibition from the experiments were analyzed with Prism software (GraphPad, San Diego, Calif., USA).

As the data herein indicate, a broad variety of compounds of formula (I), formula (II), or formula (III) were found effective as KCNH2-3.1 containing potassium channel inhibitors at low concentrations. IC₅₀ values for exemplary compounds of formula (I), formula (II), or formula (III) (see above for compound names and structures) are provided in Table 1 below. Any compound with an IC₅₀ superior or equal to 10 _{μ}M in this assay, as described above, is deemed a KCNH2-3.1 containing potassium channel inhibitor. In the Table 1 below, a single plus (+) is associated with an IC₅₀ of from about 2-10 μM; two plus signs (++) is associated with an IC₅₀ from about 500 nM to about 2 μM; and three plus signs (+++) is associated with an IC₅₀ less than about 500 nM.

TABLE 1
Activity Range of Representative Compounds of
Formula (I), (II), or (III)

| | Activity |
|---|---|
| Example | range |
| 1 | + |
| 2 | + |
| 3 | ++ |
| 4 | ++ |
| 5 | +++ |
| 6 | +++ |
| 7 | + |
| 8 | ++ |
| 9 | ++ |
| 10 | + |
| 11 | + |
| 12 | + |
| 13 | +++ |
| 14 | + |
| 15 | + |
| 16 | ++ |
| 17 | +++ |
| 18 | +++ |
| 19 | ++ |
| 20 | ++ |
| 21 | +++ |
| 22 | ++ |
| 23 | + |
| 24 | + |
| 25 | ++ |
| 26 | ++ |
| 27 | + |
| 28 | + |
| 29 | ++ |
| 30 | ++ |
| 31 | + |
| 32 | +++ |
| 33 | +++ |
| 34 | + |
| 35 | ++ |
| 36 | +++ |
| 37 | +++ |
| 38 | + |
| 39 | +++ |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Huffaker S J, Chen J, Nicodemus K K, Sambataro F, Yang F, Mattay V et al. A primate-specific, brain isoform of KCNH2 affects cortical physiology, cognition, neuronal repolarization and risk of schizophrenia. Nat Med 2009; 15(5): 509-518.

Hashimoto R, Ohi K, Yasuda Y, Fukumoto M, Yamamori H, Kamino K et al. The KCNH2 gene is associated with neurocognition and the risk of schizophrenia. World J Biol Psychiatry 2013; 14(2): 114-120.

Atalar F, Acuner T T, Cine N, Oncu F, Yesilbursa D, Ozbek U et al. Two four-marker haplotypes on 7q36.1 region indicate that the potassium channel gene HERG1 (KCNH2, Kv11.1) is related to schizophrenia: a case control study. Behav Brain Funct 2010; 6: 27.

Trudeau M C, Warmke J W, Ganetzky B, Robertson G A. HERG, a human inward rectifier in the voltage-gated potassium channel family. Science 1995; 269(5220): 92-95.

Trudeau M C, Warmke J W, Ganetzky B, Robertson G A. HERG sequence correction. Science 1996; 272(5265): 1087.

Pessia M, Servettini I, Panichi R, Guasti L, Grassi S, Arcangeli A et al. ERG voltage-gated K+channels regulate excitability and discharge dynamics of the medial vestibular nucleus neurones. J Physiol 2008; 586(Pt 20): 4877-4890.

Ji H, Tucker K R, Putzier I, Huertas M A, Horn J P, Canavier C C et al. Functional characterization of ether-à-go-go-related gene potassium channels in midbrain dopamine neurons—implications for a role in depolarization block. Eur J Neurosci 2012; 36(7): 2906-2916.

Fano S, Çalişkan G, Heinemann U. Differential effects of blockade of ERG channels on gamma oscillations and excitability in rat hippocampal slices. Eur J Neurosci 2012; 36(12): 3628-3635.

Keefe R S, Harvey P D. Cognitive impairment in schizophrenia. Handb Exp Pharmacol 2012; (213): 11-37.

Green M F, Kern R S, Braff D L, Mintz J. Neurocognitive deficits and functional outcome in schizophrenia: are we measuring the “right stuff”? Schizophr Bull 2000; 26(1): 119-136.

Mowry B J, Gratten J. The emerging spectrum of allelic variation in schizophrenia: current evidence and strategies for the identification and functional characterization of common and rare variants. Mol Psychiatry 2013; 18(1): 38-52.

Svrakic D M, Zorumski C F, Svrakic N M, Zwir I, Cloninger C R. Risk architecture of schizophrenia: the role of epigenetics. Curr Opin Psychiatry 2013; 26(2): 188-195.

Apud J A, Zhang F, Decot H, Bigos K L, Weinberger D R. Genetic variation in KCNH2 associated with expression in the brain of a unique hERG isoform modulates treatment response in patients with schizophrenia. Am J Psychiatry 2012; 169(7): 725-734.

Fatemi, S. H. and T. D. Folsom (2009). “The Neurodevelopmental Hypothesis of Schizophrenia, Revisited.” Schizophrenia Bulletin 35(3): 528-548.

Marenco, S. and D. R. Weinberger (2000). “The neurodevelopmental hypothesis of schizophrenia: Following a trail of evidence from cradle to grave.” Development and Psychopathologyl2(3): 501-527.

Pickard, B. (2011). “Progress in defining the biological causes of schizophrenia.” Expert Reviews in Molecular Medicine 13.

Titus, S. A. et al. (2009) “A new homogeneous high-throughput screening assay for profiling compound activity on the human ether-a-go-go-related gene channel” Analytical Biochemistry 394:30-38.

Although the foregoing subject matter has been described in some detail by way of illustration and example for purposes of clarity of understanding, it will be understood by those skilled in the art that certain changes and modifications can be practiced within the scope of the appended claims.

1. A compound of formula (I), formula (II), or formula (III): 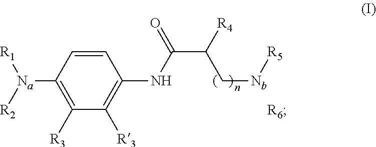
wherein:
n is 0 or 1;
R₁ and R₂ together with nitrogen atom Nₐ to which R₁ and R₂ are bound form an azepanyl or oxazepanyl ring system;
R₃ and R′₃ are each independently H or halogen;
R₄ is H or C₁-C₄ alkyl;
R₅ and R₆ together with nitrogen atom N_{b} to which R₅ and R₆ are bound form a pyrrolidinyl, piperidinyl, oxazepanyl, or azabicyclo[2.2.2]octanyl ring system, wherein:
the pyrrolidinyl ring system when present is optionally substituted with 4-fluorophenyl in the 3-position;
the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₄ alkyl in the 2-position and phenyl in the 4-position;
provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted; 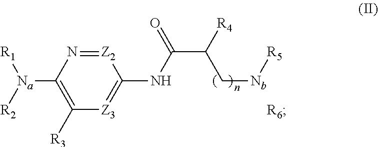
wherein:
n is 0 or 1;
Z₂ and Z₃ are each independently N or CR₇, wherein R₇ is H or halogen;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl;
or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano; or 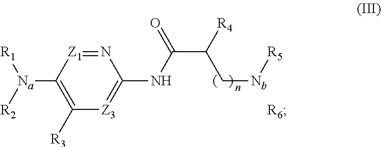
wherein:
n is 0 or 1;
Z₁ and Z₃ are each independently N or CR₇, wherein R₇ is H or halogen;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl;
or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;
and pharmaceutically acceptable salts thereof.

2. The compound of claim 1, wherein the compound is a compound of formula (II) or formula (III), and wherein R₂ is cyclohexyl or phenyl.

3. The compound of claim 1, wherein the compound is a compound of formula (II) or formula (III), and wherein R₁ and R₂ together with nitrogen atom Nₐ to which R₁ and R₂ are bound form a 6-, 7-, 8-, or 9-membered saturated cyclic, heterocyclic, or bicyclic ring system selected from azepanyl, oxazepanyl, azabicyclo[3.2.1]octanyl, and azabicyclo[3.2.2.]nonanyl.

4. The compound of claim 1, wherein the compound is a compound of formula (II) or formula (III), and wherein R₅ and R₆ together with nitrogen atom N_{b} to which R₅ and R₆ are bound form a 5-, 6-, 7-, or 8-membered saturated cyclic, heterocyclic, or bicyclic ring system selected from pyrrolidinyl, piperidinyl, oxazepanyl, azabicyclo[2.2.1]heptanyl, azabicyclo[3.2.1]octanyl, and azabicyclo[2.2.2]octanyl, wherein the pyrrolidinyl and piperidinyl can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano.

5. The compound of claim 1, wherein the compound is a compound of formula (I) selected from the following:

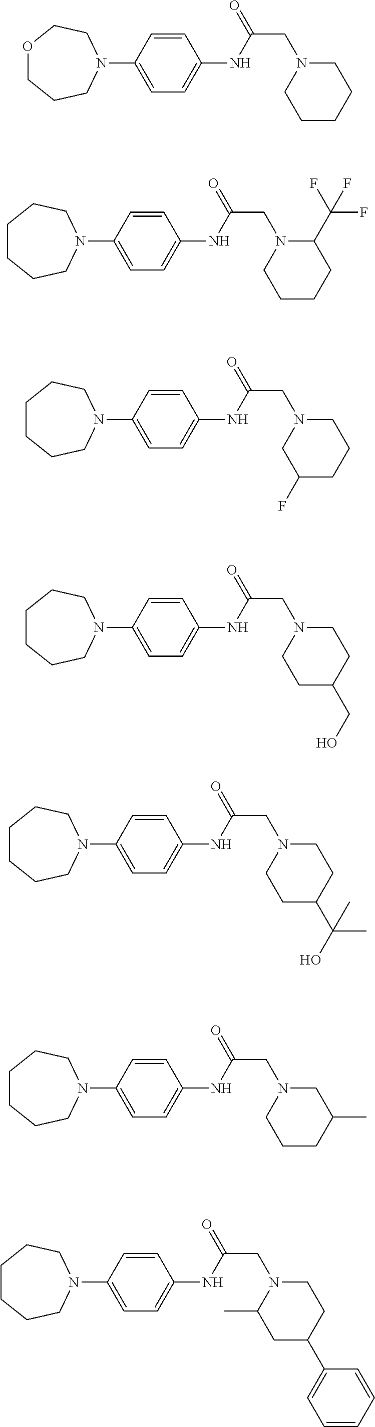

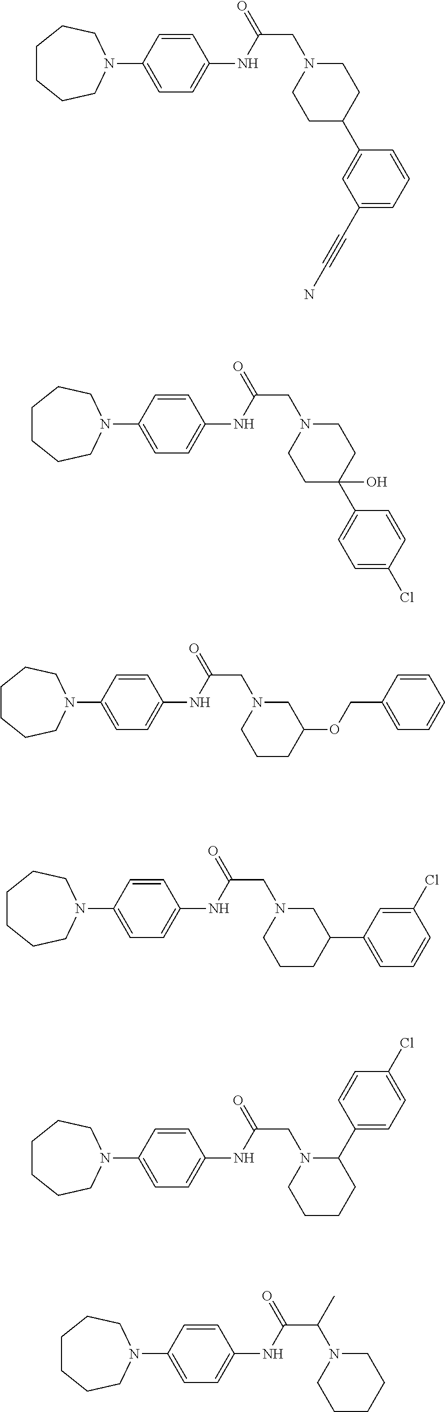 [SMILES: NC#Cc1cccc(C2CCN(CC(=O)Nc3ccc(N4CCCCCC4)cc3)CC2)c1 ; O=C(CN1CCC(O)(c2ccc(Cl)cc2)CC1)Nc1ccc(N2CCCCCC2)cc1 ; O=C(CN1CCCC(OCc2ccccc2)C1)Nc1ccc(N2CCCCCC2)cc1 ; O=C(CN1CCCC(c2cccc(Cl)c2)C1)Nc1ccc(N2CCCCCC2)cc1 ; O=C(CN1CCCCC1c1ccc(Cl)cc1)Nc1ccc(N2CCCCCC2)cc1 ; CC(C(=O)Nc1ccc(N2CCCCCC2)cc1)N1CCCCC1]

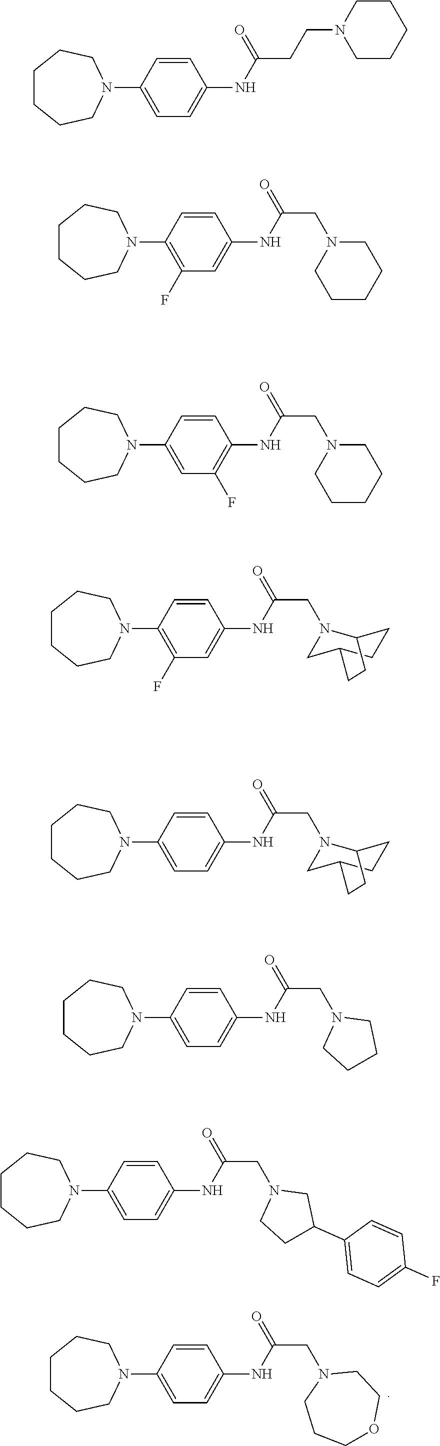

6. The compound of claim 1, wherein the compound is a compound of formula (II) and wherein:
n is 0 or 1;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl; or R₅ and R₆ together with nitrogen atom N_{b} to which R₅ and R₆ are bound form a 5-, 6-, 7-, or 8-membered saturated cyclic, heterocyclic, or bicyclic ring system, wherein the S-and 6-membered saturated cyclic ring system can optionally be substituted with C₁-C₄ alkyl, —CF₃, —(CR₈R₉)ₘ—OH, wherein m is 1, 2, 3 , or 4 and R₈ and R₉ are each independently H or C₁-C₄ alkyl, oxybenzyl, and phenyl, and wherein the phenyl can optionally be substituted with halogen or cyano;
and pharmaceutically acceptable salts thereof.

7. The compound of claim 6, wherein:
n is 0;
R₂ is cyclohexyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, selected from piperidinyl, azabicyclo[3.2.1]octanyl, and azabicyclo[3.2.2]nonanyl, wherein the piperidinyl ring system when present can optionally be substituted with C₁-C₄ alkyl;
R₄ is H;
R₅ is H or C₁-C₄ alkyl;
R₆ is selected from cyclohexyl, cycloheptyl, phenyl, and benzyl; or R₅ and R₆ together with nitrogen atom N_{b} to which R₅ and R₆ are bound form a 6-, 7-, or 8-membered saturated cyclic, heterocyclic, or bicyclic ring system selected from piperidinyl, azabicyclo[3.2.1]octanyl, azabicyclo[2.2.2]octanyl, and azabicyclo[2.2.1]heptanyl.

8. The compound of claim 7, wherein the compound is selected from the group consisting of:

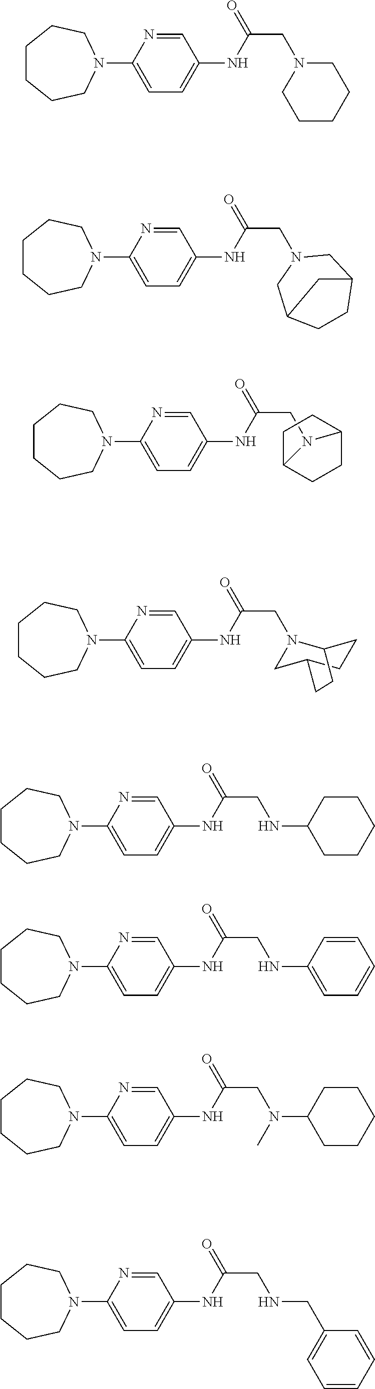

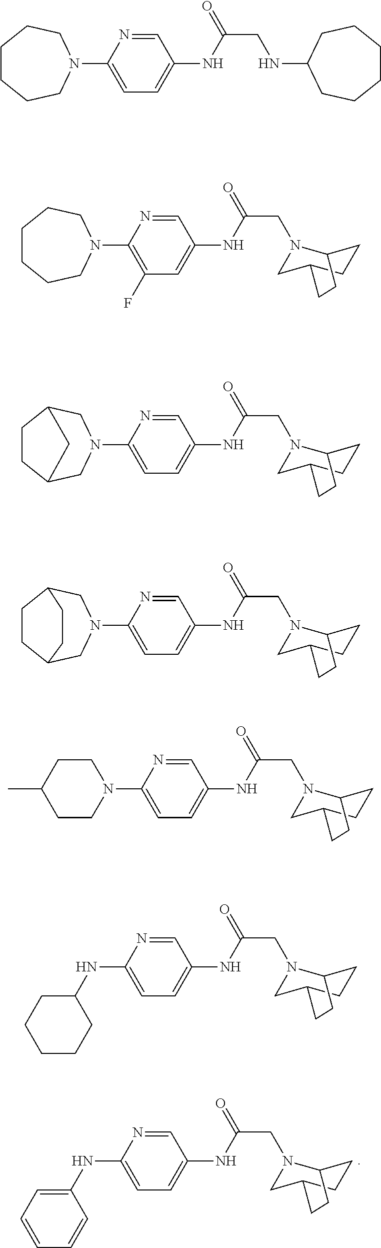

9. The compound of claim 1, wherein the compound is a compound of formula (III) and wherein:
n is 0 or 1;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl;
or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;
and pharmaceutically acceptable salts thereof.

10. The compound of claim 9, wherein:
n is 1;
R₁ and R₂ together with nitrogen atom Nₐ to which R₁ and R₂ are bound form an azepanyl ring system;
R₃ and R₄ are each H;
R₅ and R₆ together with nitrogen atom N_{b} to which R₅ and R₆ are bound form a piperidinyl ring system; and the compound is selected from:

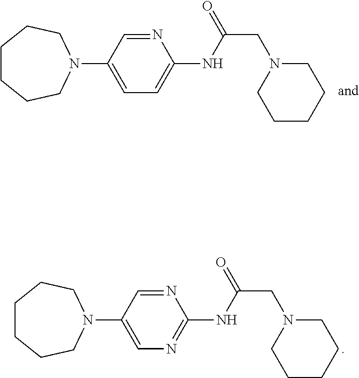 [SMILES: O=C(CN1CCCCC1)Nc1ccc(N2CCCCCC2)cn1 ; O=C(CN1CCCCC1)Nc1ncc(N2CCCCCC2)cn1]

11. A pharmaceutical composition comprising at least one compound of claim 1 and a pharmaceutically acceptable carrier.

12. The pharmaceutical composition of claim 12, further comprising at least one additional therapeutic agent.

13. The pharmaceutical composition of claim 12, wherein the at least one additional therapeutic agent is selected from the group consisting of one or more antipsychotic agents.

14. The pharmaceutical composition of claim 13, wherein the one or more antipsychotic agents is selected from olanzapine, risperidone, paliperidone, aripriprazole, clozapine, perphenazine, quetiapine, haloperidol, lurasidone, and combinations thereof.

15. A method for treating a neurological or psychiatric disorder, or treating symptoms associated with a neurological or psychiatric disorder, the method comprising administering to a subject in need of treatment thereof a therapeutically effective amount of a compound of formula (I), formula (II), or formula (III), or a pharmaceutically acceptable salt thereof: 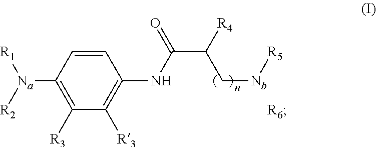
wherein:
n is 0 or 1;
R₁ and R₂ together with nitrogen atom Nₐ to which R₁ and R₂ are bound form an azepanyl or oxazepanyl ring system;
R₃ and R′₃ are each independently H or halogen;
R₄ is H or C₁-C₄ alkyl;
R₅ and R₆ together with nitrogen atom N_{b} to which R₅ and R₆ are bound form a pyrrolidinyl, piperidinyl, oxazepanyl, or azabicyclo[2.2.2]octanyl ring system, wherein:
the pyrrolidinyl ring system is optionally substituted with 4-fluorophenyl in the 3-position;
the piperidinyl ring system when present is substituted with one of —CF₃ or halophenyl in the 2-position; halogen, halophenyl, benzyloxyl, or C₁-C₄ alkyl in the 3-position; cyanophenyl, halophenyl, hydroxyl, or —(CR₈R₉)ₘ—OH, wherein m is 1, 2, 3, or 4 and R₈ and R₉ are each independently H or C₁-C₄ alkyl, in the 4-position; or a combination of C₁-C₈ alkyl in the 2-position and phenyl in the 4-position;
provided that if: (i) n is 2; (ii) R₃ or R′₃ are halogen; (iii) R₄ is C₁-C₄ alkyl; or (iv) R₁ and R₂ together with nitrogen atom Nₐ to which R₁ and R₂ are bound form an oxazepanyl ring system, then the piperidinyl ring system when present can be unsubstituted; 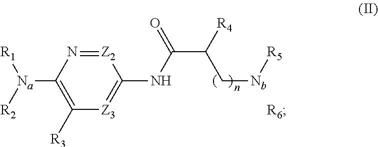
wherein:
n is 0 or 1;
Z₂ and Z₃ are each independently N or CR₇, wherein R₇ is H or halogen;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl;
or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano; or 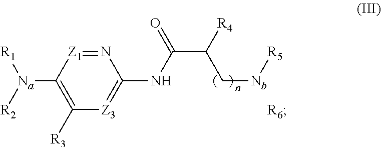
wherein:
n is 0 or 1;
Z₁ and Z₃ are each independently N or CR₇, wherein R₇ is H or halogen;
R₁ is H;
R₂ is cycloalkyl or phenyl; or R₁ and R₂ together with nitrogen atom Nₐ to which R₁ and R₂ are bound form a 6-, 7-, 8-, or 9-membered saturated cyclic, heterocyclic, or bicyclic ring system, wherein the 6-membered saturated cyclic ring system can optionally be substituted with C₁-C₄ alkyl;
R₃ is H or halogen;
R₄ and R₅ are each independently H or C₁-C₄ alkyl;
R₆ is selected from cycloalkyl, phenyl, and benzyl;
or R₅ and R₆ together with nitrogen atom N_{b} to which R₅ and R₆ are bound form a 5-, 6-, 7-, or 8-membered saturated cyclic, heterocyclic, or bicyclic ring system, wherein the 5- and 6-membered saturated cyclic ring system can optionally be substituted with C₁-C₄ alkyl, —CF₃, —(CR₈R₉)ₘ—OH, wherein m is 1, 2, 3, or 4 and R₈ and R₉ are each independently H or C₁-C₄ alkyl, oxybenzyl, and phenyl, and wherein the phenyl can optionally be substituted with halogen or cyano;
and pharmaceutically acceptable salts thereof.

16. The method of claim 15, wherein the neurological or psychiatric disorder is selected from schizophrenia, major depression, a depressive phase of bipolar disorder, attention deficit disorder, attention deficit/hyperactivity disorder, substance dependency, and increased appetite associated with smoking cessation or antipsychotic use.

17. The method of claim 16, wherein the neurological or psychiatric disorder is schizophrenia.

18. The method of claim 15, wherein administering to the subject a therapeutically effective amount of a compound of formula (I), or a pharmaceutically acceptable salt thereof, inhibits one or more Kv11.1-3.1 containing potassium channels.
